(19) United States
(12) Patent Application Publication
(10) Pub. No.: US 2023/0347353 A1
(43) Pub. Date: Nov. 2, 2023

(54) SYSTEMS AND METHODS FOR ANALYZING A BIOLOGICAL SAMPLE

(71) Applicant: COMBINATI INCORPORATED, Carlsbad, CA (US)

(72) Inventors: Ju-Sung HUNG, Carlsbad, CA (US); Lingxia JIANG, Boyds, MD (US); Robert LIN, Palo Alto, CA (US)

(21) Appl. No.: 18/002,310

(22) PCT Filed: Jun. 21, 2021
(86) PCT No.: PCT/US2021/038206

§ 371 Date: Dec. 19, 2022

Related U.S. Application Data
(60) Provisional application No. 63/042,353, filed on Jun. 22, 2020.

(51) Int. Cl. B01L 7/00; C12Q 1/6853

(52) U.S. Cl. CPC B01L 7/525; C12Q 1/6853; B01L 2200/10; B01L 2300/0654; B01L 2300/0819; B01L 2300/0861; B01L 2300/1822; B01L 2300/0893; B01L 2300/0663

(57) ABSTRACT

The present disclosure provides methods and systems for nucleic acid identification. Identification of a nucleic acid molecule may include generating, in a plurality of chambers, a plurality of double-stranded nucleic acid molecules, denaturing the double-stranded nucleic acid molecules, and detecting signals of the denaturation to generate one or more denaturation profiles. The one or more denaturation profiles may be usable to identify nucleic acid molecules. The methods and system described herein may provide for identification of multiple nucleic acid molecules from a single analysis.

### CROSS-REFERENCE

This application is related to PCT Application Serial No. PCT/US2021/038206 filed Jun. 21, 2021, which claims the benefit of U.S. Provisional Patent Application No. 63/042,353, filed Jun. 22, 2020, which is entirely incorporated herein by reference.

### GOVERNMENT INTEREST STATEMENT

This invention was made with government support under Small Business Innovation Research grant number 1R43CA221597-01A1 awarded by the National Cancer Institute. The U.S. government has certain rights in the invention.

### BACKGROUND

Microfluidic devices are devices that contain structures that handle fluids on a small scale. Typically, a microfluidic device operates on a sub-millimeter scale and handles microliters, nano-liters, or smaller quantities of fluids. One application of microfluidic devices is in analyte analysis, e.g., digital polymerase chain reaction (dPCR). Microfluidic devices with multiple partitions may be useful for dPCR. Unlike quantitative real-time PCR (qPCR) where templates are quantified by comparing the rate of PCR amplification of an unknown sample to the rate for a set of known qPCR standards, dPCR may provide higher sensitivity, better precision, and greater reproducibility.

For genomic researchers and clinicians, dPCR is particularly powerful in rare mutation detection, quantifying copy number variants, and Next Gen Sequencing library quantification. The potential use in clinical settings for liquid biopsy with cell free DNA and viral load quantification further increases the value of dPCR technology. Existing dPCR solutions have used elastomeric valve arrays, silicon through-hole approaches, and microfluidic encapsulation of droplets in oil. Despite the growing number of available dPCR platforms, dPCR has been at a disadvantage when compared to the older qPCR technology which relies on counting the number of PCR amplification cycles. The combination of throughput, ease of use, performance and cost are the major barriers for gaining adoption in the market for dPCR.

### SUMMARY

Provided herein are methods and systems that may be useful for detecting, identifying, or quantifying an analyte or multiple analytes. The present disclosure provides methods, systems, and devices for sample preparation, nucleic acid amplification, analyte analysis, multiplex analyte analysis, or any combination thereof. The method, systems, and devices described herein may permit detection, identification, or quantification of analytes at a reduced cost or complexity as compared to other systems and methods.

In an aspect, the present disclosure provides a method for nucleic acid identification, comprising: (a) using a plurality of nucleic acid molecules to generate, in a plurality of chambers, a plurality of double-stranded nucleic acid molecules, wherein: (i) a first subset of the plurality of double-stranded nucleic acid molecules comprises a first double-stranded nucleic acid molecule comprising a first sequence corresponding to a first nucleic acid molecule of the plurality of nucleic acid molecules and an added sequence; and (ii) a second subset of the plurality of double-stranded nucleic acid molecules comprises a second double-stranded nucleic acid molecule comprising a second sequence corresponding to a second nucleic acid molecule of the plurality of nucleic acid molecules and does not comprise the added sequence; (b) denaturing double-stranded nucleic acid molecules of the plurality of double-stranded nucleic acid molecules; (c) detecting signals indicative of the denaturing to generate a plurality of denaturation profiles, wherein: i. a first denaturation profile of the plurality of denaturation profiles is derived from denaturation of the first double-stranded nucleic acid molecule; ii. a second denaturation profile of the plurality of denaturation profiles is derived from denaturation of the second double-stranded nucleic acid molecule; and iii. the first denaturation profile and the second denaturation profile are different; and (d) processing the plurality of denaturation profiles to identify a nucleic acid molecule of the plurality of nucleic acid molecules.

In some embodiments, the method further comprises, prior to (a), providing the plurality of nucleic acid molecules and a plurality of forward primers to the plurality of chambers. In some embodiments, the plurality of forward primers comprises (i) a first forward primer comprising a first region complementary to at least a portion of the first nucleic acid molecule and a second region that is not complementary to the first nucleic acid molecule and corresponds to the added sequence and (ii) a second forward primer complementary to at least a portion of the second nucleic acid molecule. In some embodiments, the plurality of forward primers are not universal primers. In some embodiments, the method further comprises, prior to (a), subjecting the plurality of forward primers to primer extension reactions to generate a plurality of first extension products. In some embodiments, the method further comprises, prior to (a), contacting the plurality of first extension products with a plurality of reverse primers. In some embodiments, the plurality of reverse primers are universal primers. In some embodiments, the method further comprises, prior to (a), subjecting the plurality of reverse primers to primer extension reactions to generate a plurality of second extension products. In some embodiments, the plurality of second extension products are the plurality of double-stranded nucleic acid molecules.

In some embodiments, the method further comprises imaging at least a portion of the plurality of chambers to detect the signals. In some embodiments, the method further comprises imaging the plurality of chambers to detect the signals. In some embodiments, the method further comprises subjecting the plurality of double-stranded nucleic acid molecules to controlled heating to denature the double-stranded nucleic acid molecules. In some embodiments, the double-stranded nucleic acid molecules comprise intercalating dyes from which the signals are derived. In some embodiments, the double-stranded nucleic acid molecules comprise a plurality of different intercalating dyes from which the signals are derived. In some embodiments, the signals are optical signals. In some embodiments, a chamber of the plurality of chambers has a volume of less than or equal to about 500 picoliters. In some embodiments, the volume of the chamber is less than or equal to about 250 picoliters. In some embodiments, the plurality of chambers comprises greater than or equal to about 1,000 chambers. In some embodiments, the plurality of chambers comprises greater than or equal to about 10,000 chambers.

In another aspect, the present disclosure provides a system for nucleic acid identification, comprising: a detection unit configured to collect and process signals for identification of nucleic acid molecules; and one or more processors operatively coupled to the detection unit, wherein the one or more processors are individually or collectively programmed or otherwise configured to: (i) use a plurality of nucleic acid molecules to generate, in a plurality of chambers, a plurality of double-stranded nucleic acid molecules, wherein: (i) a first subset of the plurality of double-stranded nucleic acid molecules comprises a first double-stranded nucleic acid molecule comprising a first sequence corresponding to a first nucleic acid molecule of the plurality of nucleic acid molecules and an added sequence; and (ii) a second subset of the plurality of double-stranded nucleic acid molecules comprises a second double-stranded nucleic acid molecule comprising a second sequence corresponding to a second nucleic acid molecule of the plurality of nucleic acid molecules and does not comprise the added sequence; (ii) denature double-stranded nucleic acid molecules of the plurality of double-stranded nucleic acid molecules; (iii) detect signals indicative of the denaturing to generate a plurality of denaturation profiles, wherein: (A) a first denaturation profile of the plurality of denaturation profiles is derived from denaturation of the first double-stranded nucleic acid molecule; (B) a second denaturation profile of the plurality of denaturation profiles is derived from denaturation of the second double-stranded nucleic acid molecule; and (C) the first denaturation profile and the second denaturation profile are different; and (iv) processing the plurality of denaturation profiles to identify a nucleic acid molecule of the plurality of nucleic acid molecules.

In some embodiments, a chamber of the plurality of chambers has a volume of less than or equal to about 500 picoliters. In some embodiments, the volume of the chamber is less than or equal to about 250 picoliters. In some embodiments, the plurality of chambers comprises greater than or equal to about 1,000 chambers. In some embodiments, the plurality of chambers comprises greater than or equal to about 10,000 chambers. In some embodiments, the detection unit is configured to image at least a portion of the plurality of chambers. In some embodiments, the detection unit is configured to image the plurality of chambers. In some embodiments, the detection unit comprises a camera with a field of view of greater than or equal to about 15 millimeters (mm) by about 15 mm. In some embodiments, the field of view is greater than or equal to about 50 mm by about 75 mm. In some embodiments, the detection unit comprises a camera comprising a complementary metal-oxide-semiconductor (CMOS) sensor. In some embodiments, the detection unit further comprises a telecentric lens disposed between the camera and the plurality of chambers. In some embodiments, the detection unit comprises an optical unit configured to collect optical signals. In some embodiments, the optical unit comprises greater than or equal to four channels, each channel configured to collect a different wavelength of light.

In some embodiments, the system is configured to receive a substrate comprising a plurality of chamber arrays, and wherein a chamber array of the plurality of chamber arrays comprises the plurality of chambers. In some embodiments, the substrate comprises at least four chamber arrays. In some embodiments, the chamber array is fluidically isolated from another chamber array. In some embodiments, the system is configured to receive a plate, and wherein the plate is configured to retain a plurality of substrates comprising the substrate. In some embodiments, the system further comprises a thermal unit operatively coupled to the one or more processors, wherein the thermal unit is configured to control a temperature of the plurality of chambers. In some embodiments, the one or more processors directs the thermal unit to subject the plurality of chambers to controlled heating to denature the double-stranded nucleic acid molecules. In some embodiments, the thermal unit comprises a thermoelectric temperature control unit.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an example comparison of polymerase chain reaction, quantitative polymerase chain reaction, and digital polymerase chain reaction nucleic acid analysis;

FIG. 2 shows an example workflow for processing as sample using an example integrated digital polymerase chain reaction (dPCR) platform;

FIGS. 3A-3G show example consumables for dPCR and example data generated from an example system; FIG. 3A shows an example microfluidic device comprising multiple microfluidic arrays; FIG. 3B shows an scanning electron microscope image of an example consumable, FIG. 3C shows an example of sample digitization and consistency across microfluidic arrays; FIG. 3D shows an example of four-channel imaging of an example consumable; FIG. 3E shows an example assay result from an example consumable and integrated system; FIG. 3F shows another example assay result from an example consumable and integrated system; FIG. 3G shows another example assay result from an example consumable and integrated system;

FIGS. 4A and 4B show examples of real-time analysis of dPCR partitions; FIG. 4A shows example real-time PCR curves for positive partitions; FIG. 4B shows example real-time PCR curves for positive curves for an assay prone to non-specific amplification;

FIG. 5 schematically illustrates an example process for nucleic acid identification

FIG. 6 shows an example of workflow for determining method and system performance;

FIGS. 7A-7C show an example process for demonstrating system performance; FIG. 7A shows an example sample preparation workflow to quantify and purify nucleic acid molecules within a sample; FIG. 7B shows an example of sample partitioning and denaturation profiles; FIG. 7C shows an example database comparison of melt curves to identify analytes within the sample;

FIG. 8 shows example denaturation profiles for a panel-based respiratory pathogen;

FIGS. 9A-9C show example design parameters for an example integrated system; FIG. 9A shows example target parameters for a whole plate imager; FIG. 9B shows an example microfluidic array and field of view; FIG. 9C shows an example image generated from an example integrated system;

FIG. 10 shows an example optical module for imaging;

FIG. 11 shows an example integrated system for dPCR;

FIG. 12 shows a computer system that is programmed or otherwise configured to implement methods provided herein;

FIG. 13 shows example fluorescent images acquired at four different temperatures for an example assay;

FIG. 14 shows example melt profiles for three different sample targets; and

FIGS. 15A-15E show example melt curve analysis for microorganism species identification; FIG. 15A shows example melt profiles for a library of bacterial species; FIG. 15B shows example melt profiles for a select number of Bacillus bacterial species; FIG. 15C shows example melt profiles for a select number of Staphylococcus species; FIG. 15D shows a heat map of bacterial species organized by phylum; and FIG. 15E shows another example heat map.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “sample,” as used herein, generally refers to any sample containing or suspected of containing an analyte. For example, a sample can be a biological sample containing one or more analytes. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood. In such instance, the sample can include cell-free DNA, cell-free RNA, proteins, metabolites, or any combination thereof. In some examples, the sample can include circulating tumor cells, cancer biomarkers, or both. In some examples, the sample is an environmental sample (e.g., soil, waste, ambient air and etc.), industrial sample (e.g., samples from any industrial processes), and food samples (e.g., dairy products, vegetable products, and meat products). The sample may be processed prior to loading into a microfluidic device. For example, the sample may be processed to lyse cells, purify proteins, or to include reagents. Alternatively, or in addition to, the sample may not be processed prior to loading into a microfluidic device.

As used herein, the term “fluidic” or “microfluidic” may be used interchangeable and generally refer to a chip, area, device, article, or system including at least one channel in fluid communication with an array of chambers. The channel may have a cross-sectional dimension less than or equal to about 10 millimeters (mm), less than or equal to about 5 mm, less than or equal to about 4 mm, less than or equal to about 3 mm, less than or equal to about 2 mm, less than or equal to about 1.5 mm, less than or equal to about 1 mm, less than or equal to about 750 micrometers (μm), less than or equal to about 500 μm, less than or equal to about 250 μm, less than or equal to about 100 μm, or less. The chambers may have a volume of less than or equal to about 100 microliters (μL), 50 μL, 25 μL, 10 μL, 5 μL, 1 μL, 500 nanoliters (nL), 250 nL, 100 nL, 50 nL, 25 nL, 10 nL, 5 nL, 1 nL, 500 picoliters (pL), 250 pL, 100 pL, 50 pL, 25 pL, 10 pL, 5 pL, 1 pL, or less.

As used herein, the term “fluid,” generally refers to a liquid or a gas. A fluid cannot maintain a defined shape and flows during an observable time frame to fill the container into which it is put. Thus, the fluid may have any suitable viscosity that permits flow. If two or more fluids are present, each fluid may be independently selected among any fluids (e.g., liquids, gases, and the like).

As used herein, the term “partition,” generally refers to a division into or distribution into portions or shares. For example, a partitioned sample is a sample that is isolated from other samples. Examples of structures that enable sample partitioning include wells, chambers, droplets, or any combination thereof.

As used herein, the terms “pressurized off-gassing” or “pressurized degassing” may be used interchangeable and generally refer to removal or evacuation of a gas (e.g., air, nitrogen, oxygen, etc.) from a channel or chamber of the device (e.g., microfluidic device) to an environment external to the channel or chamber through the application of a pressure differential. The pressure differential may be applied between the channel or chamber and the environment external to the channel or chamber. The pressure differential may be provided by the application of a pressure source to one or more inlets to the device or application of a vacuum source to one or more surfaces of the device. Pressurized off-gassing or pressurized degassing may be permitted through a film or membrane covering one or more sides of the channel or chamber.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Provided herein are methods and systems that may be useful for detecting, identifying, or quantifying an analyte or multiple analytes (e.g., nucleic acid molecules). The present disclosure provides methods, systems, and devices for sample preparation, nucleic acid amplification, analyte analysis, multiplex analyte analysis, or any combination thereof. The method, systems, and devices described herein may permit detection, identification, or quantification of analytes at a reduced cost or complexity as compared to other systems and methods.

Polymerase Chain Reaction (PCR), which describes in vitro amplification of a specific small amount of nucleic acid molecule in a sample to a larger quantity (e.g., large enough to study). Quantitative PCR (qPCR) may be used to perform relative quantification of nucleic acid samples. Digital PCR (dPCR) may be used for rare allele detection in a variety of platforms from 384 well plates to droplet-based platforms using water-in-oil emulsions. Digital PCR may leverage sample dilution to generate a large number of partitions with less than one nucleic acid template per partition. The total number of templates may then be quantified by counting the number of partitions in which the template is successfully amplified. To account for partitions having more than one template, Poisson statistics may be applied. Unlike qPCR where templates are quantified by comparing the rate of PCR amplification of an unknown sample to the rate for a set of known standards, quantification by dPCR may have higher sensitivity, better precision, and greater reproducibility.

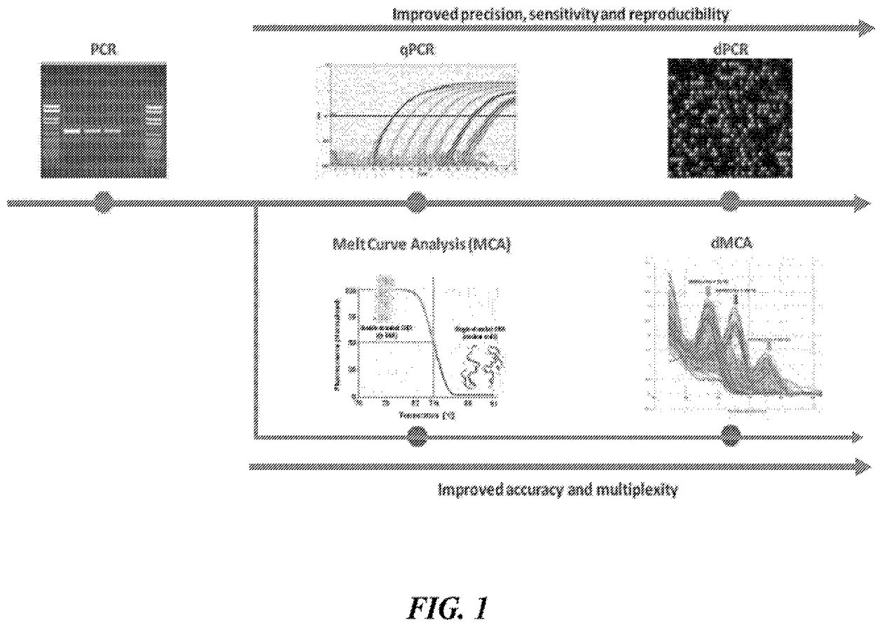

In some examples, PCR platforms may be used in tandem with other techniques for detection, identification, or quantification of analytes (e.g., nucleic acid molecules). For example, melt curve analysis (MCA), may use intercalating fluorescent dye to assess denaturation characteristics of double-stranded nucleic acid molecules (e.g., PCR products or amplicons) during heating. With precise temperature control, high resolution melt (HRM) may detect small differences in nucleic acid sequences, such as, for example, methylation analysis, mutation scanning, and genotyping. Probe-based melt technologies may further complement sequencing and improved multiplexing. Similar to dPCR, by diluting the samples down to less than one template per partition, the melt curves of different amplicons may be clearly distinguished in each partition, avoiding the averaging effect of melt curve analysis in bulk solution. Digital melt curve analysis (dMCA) may be useful for a variety of applications, for example, including for bacterial deoxyribonucleic acid (DNA) sequence profiling, facile profiling of molecular heterogeneity for cancer liquid biopsy, and Kirsten Ras 1 (KRAS) genotyping. As shown in FIG. 1, by leveraging the temperature-dependent dissociation of double-stranded nucleic acid molecules, dMCA may offer another dimension (e.g., temperature) to further improve quantification accuracy and multiplexity for nucleic acid identification and quantification.

There may be a number of challenges to implementing and integrating MCA with a digital platform (e.g., dPCR platform). For example, integration of reagent digitization, effective and consistent thermal cycling, and imaging may be challenging. There are many considerations for effective implementation of consumables for dMCA, such as a large number of partitions (e.g., greater than 10,000) to provide high statistical confidence for quantification, high throughput via integration with laboratory automation equipment, low-cost and highly scalable manufacturing, and evaporation prevention. Integration of melt-based chemistries with the consumable may also be challenging. For example, the use of select intercalating dyes in combination with select plastics may result in non-specific adsorption of the dyes to the plastic material.

Current approaches for MCA may not address the challenges of dMCA. For example, the use of silicon through-hole arrays for simultaneous thermal cycling and imaging may have limited throughput (e.g., may be limited to one sample per experiment) and poor manufacturability in terms of the cost of semiconductor processing. Alternative approaches may use microfluidic devices formed at least partially of polydimethylsiloxane (PDMS). However, microfluidic devices formed from PDMS may have poor manufacturing reproducibility, may not prevent reagent evaporation (e.g., significant reagent may evaporate during thermal cycling), and limited chemistry compatibility.

The present disclosure provides methods and systems to address the challenges of dMCA. The methods and systems described herein may use a microfluidic array for partitioning samples. The microfluidic array may include dead-ended, injection-molded microchamber arrays sealed with a semi-permeable or pressure permeable membrane for reagent partitioning. See, for example, International Patent Application No. PCT/US2017/025873, filed Apr. 4, 2017, International Patent Application No. PCT/US2017/062078, filed Nov. 16, 2017, International Patent Application No. PCT/US2019/065287, filed Dec. 9, 2019, each of which is incorporated herein by reference in its entirety. The methods and systems described herein may provide digital melt curve analysis with similar ease of use as qPCR, low cost per data point, and high throughput.

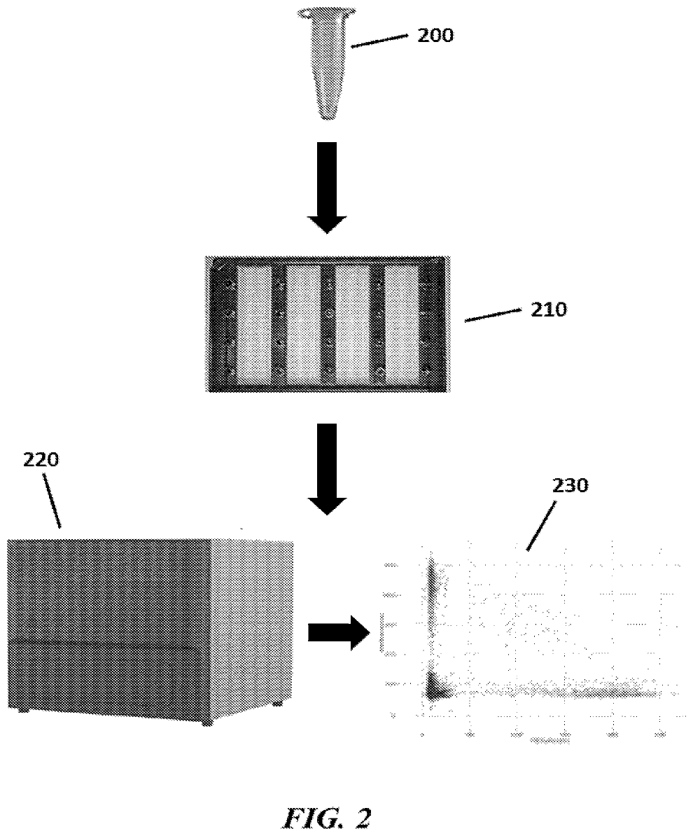

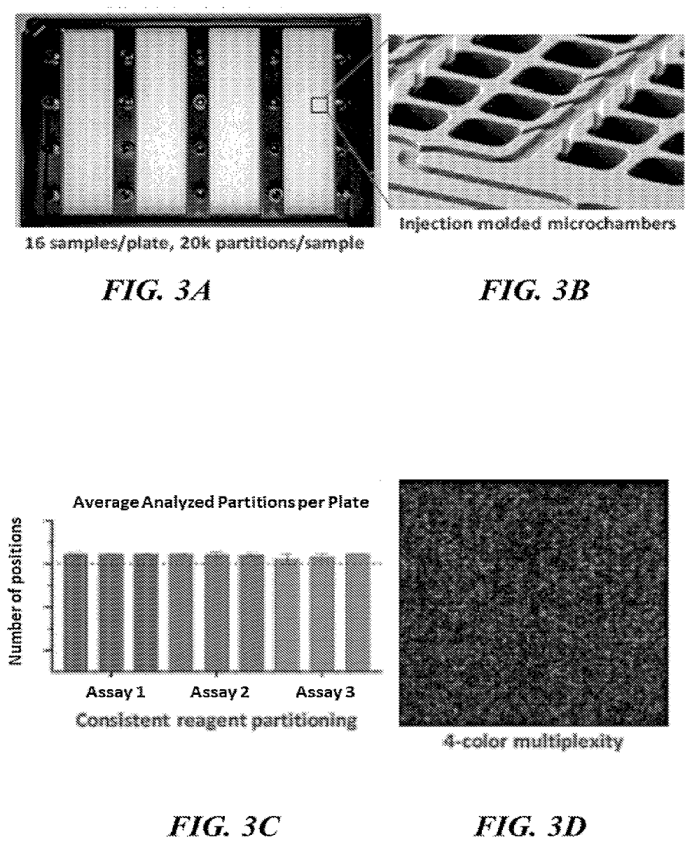

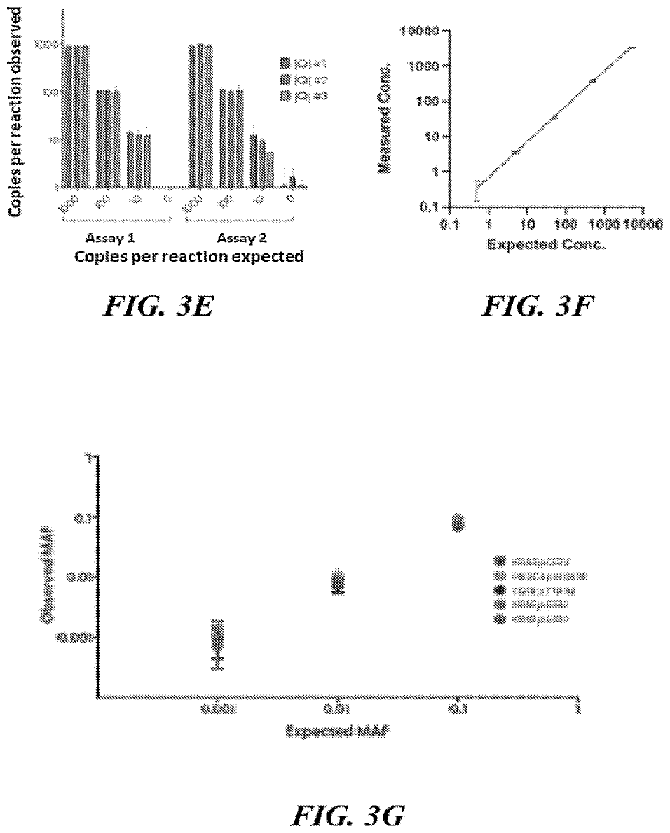

The systems described herein may include an integrated dPCR platform. The dPCR platform may integrate the various processes used for dPCR, for example, partitioning of reagents, thermal cycling of reaction mixtures and acquisition of data, into a single instrument. This may permit the dPCR workflow to replicate that of qPCR, with instrumentation architect comprising improved reliability and lowered cost as compared to qPCR instrumentation. For example, the process may be fully automated such that a user loads the reaction mixture into the consumable plate, places the plate into the instrument, and begins a sample processing program, as shown in FIG. 2. For example, a sample may be provided in a solution 200. The sample may include a plurality of nucleic acid molecules. The solution 200 may be provided to a microfluidic device 210 via a fluid flow system comprising a pneumatic module or other fluid handling modules. The microfluidic device 210 may be a microfluidic array that provides partitioning of the sample. The microfluidic device 210 may include a single array for partitioning the sample or multiple arrays for partitioning the sample. The microfluidic device 210 may be loaded into an analysis system 220. The analysis system 220 may be an fully integrated analysis platform configured to process and analyze the sample. Alternatively, or in addition to, the analysis system 220 may include the microfluidic device 210 and the sample may be provided to the analysis system for partitioning into the fluidic device 210. Subsequent to sample processing, the analysis system 220 may analyze the sample (e.g., by using a detection unit to collect signals derived from the sample) and process the signals to generate one or more data outputs 230. The system may be a benchtop system (e.g., with a housing size of about 2 foot (ft) by 2 ft by 2 ft). The system may include a detection unit comprising an optical module that permits scanning through a microfluidic device comprising multiple arrays of partitions (e.g., a device comprising sixteen arrays of partitions). FIG. 3A shows an example microfluidic device comprising sixteen microfluidic arrays with 20,000 partitions (e.g., chambers) per array. A microfluidic array with sixteen arrays may permit processing of sixteen different samples simultaneously. The detection unit may permit real-time detection (e.g., imaging) during sample processing and analysis.

The combination of the microfluidic array and integrated analysis platform may permit simple sample processing and analysis and provide enhanced consistency as compared to other methods and systems. For example, unlike platforms that leverage stochastic microfluidic droplet generation mechanisms to partition the bulk reaction or relying on positive fluid displacement, the microfluidic array consumable may use a fixed, injection-molded microchamber array that includes both the precise volume and total number of the nanoliter-volume partitions as shown in the SEM image in FIG. 3B. Using the device geometry to precisely define the partitions may permit the platform greater flexibility and robustness against reagent variations. For example, FIG. 3C shows an example of the total analyzed partitions from three experiments performed with three different master mix-assay combinations. Across nine plates (144 arrays total), the averaged total analyzed partition per sample is 20,412 with a coefficient of variation of 0.68%. This highly consistent partition number across master mixes, assays and runs may illustrate the stability of the partitioning process and site-to-site consistency. In an example, the instrument may support four different optical channels, as shown in FIG. 3D, which may permit analysis and quantification of four different targets within a sample. As the consumable may be made from thermoplastics (e.g., Cyclo-Olefin-Polymer), which may act as a moisture barrier, the may be little to no reagent evaporation throughout the process. Additional example data is shown in FIGS. 3E-3G. FIG. 3E shows example using an example system for wet-lab verified data including reference material dynamic range quantification. FIG. 3F shows an example of using an example system for BCR-Abl EuroStandard quantification from 10,000 copies per microliter (copies/μL) down to 0.1 copy/μL. FIG. 3G shows an example of using an example system for TaqMan dPCR liquid biopsy rare allele fraction assay down to 0.1%.

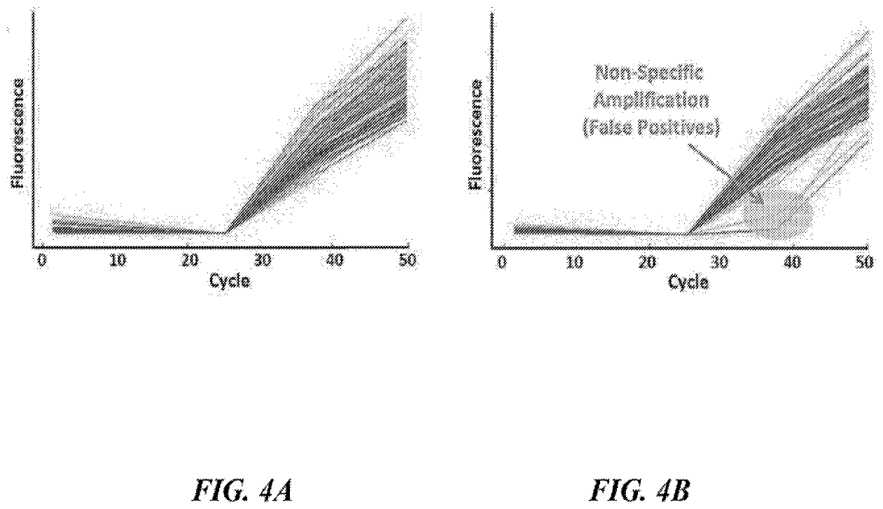

The methods and systems described herein may further provide dPCR platforms that permit fluorescence analysis of dPCR partitions at any point during traditional PCR thermal cycling or during post-PCR melt. This is a large benefit over current dPCR platforms that provide for end-point analysis of dPCR droplets or partitions and not real-time measurements. For example, non-specific amplification and contaminants may result in false positive partitions. In dPCR platforms that use end-point analysis, a false positive cannot be distinguished from a true positive. Alternatively, in the system describe herein, real-time PCR dynamics of individual partitions may be monitored permitting differentiation between false true positives. For example, if the fluorescence in a positive partition does not fit the expected PCR amplification dynamics, then this partition may be considered a false positive and excluded from analysis. FIG. 4A shows amplification dynamics of an example SMA assay from an example integrated system. In the example, fluorescent images are acquired at pre-determined cycles during PCR rather than every cycle. By decreasing a number of images taken, the analysis and complexity of the analysis may be reduced. Individual lines of FIG. 4A represent the fluorescence for an individual partition across cycles. FIG. 4B shows an example assay with a problematic non-specific amplification. Discrete real-time analysis may reveal partitions with late cycle amplification dynamics. These partitions may have non-specific amplification and, thus, may be considered false positives and removed from the analysis. The use of real-time dPCR analysis may also be used to eliminate the need for arbitrary thresholding. For example, partitions with expected PCR amplification dynamics may be considered positive and all others may be considered be negative. As such, real-time processing of dPCR dynamics may both improve and automate overall dPCR analysis.

In an example, the methods and systems described herein may be used for digital biology (e.g., single cell, single protein, and single nucleic acid analysis). Digital biology has significantly improved the resolution of science. In digital genomics, partitioning reagents may eliminate the need of standard curves, thus greatly improving reproducibility. Moreover, background materials such as inhibitors may also be partitioned, improving the reaction specificity and sensitivity. Current dPCR platforms may leverage fluorescent probes for multiplexing, but do not support digital melt curve analysis. By adding melt curve analysis capability to the dPCR platform and leveraging the temperature dependent nature of double-stranded nucleic acid denaturation, the quantification accuracy may be enhanced as amplicon melt signatures can be assessed to eliminate false positives and signals from non-specific amplification. In addition, another modality—melting temperatures (Tₘ)—may be added to multiplex different targets, further reduces the cost per data point, and allow quantification of a panel of genomic biomarkers with high accuracy, precision, sensitivity and reproducibility. As such, the methods and systems described herein may provide high performance, are easy to use, and affordable digital biology platforms, which in turn may accelerate adoption of digital genomics and positively impact healthcare.

### Methods for Nucleic Acid Identification

In an aspect, the present disclosure provides methods for nucleic acid identification. The method may include using a plurality of nucleic acid molecules to generate, in a plurality of chambers, a plurality of double-stranded nucleic acid molecules, denaturing the double-stranded nucleic acid molecules, detecting signals indicative of the denaturation of the double-stranded nucleic acid molecules to generate a plurality of denaturation profiles, and processing the denaturation profiles to identify at least one nucleic acid molecule. The plurality of double-stranded nucleic acid molecules may include a first subset of double-stranded nucleic acid molecules and a second subset of double-stranded nucleic acid molecules. The first subset of double-stranded nucleic acid molecules may include a first double-stranded nucleic acid molecule comprising a first sequence corresponding to a first nucleic acid molecule and an added sequence. The plurality of double-stranded nucleic acid molecules may include a second subset of double-stranded nucleic acid molecules comprising a second double-stranded nucleic acid molecule. The second double-stranded nucleic acid molecule may comprise a second sequence corresponding to a second nucleic acid molecule and does not include an added sequence. The first double-stranded nucleic acid molecule may generate a first denaturation profile upon denaturation and the second double-stranded nucleic acid molecule may generate a second denaturation profile upon denaturation. The first and second denaturation profiles may be different and distinguishable. The added sequence may modulate the denaturation profile of the first double-stranded nucleic acid molecule to permit or enhance differentiation between the first and second denaturation profiles.

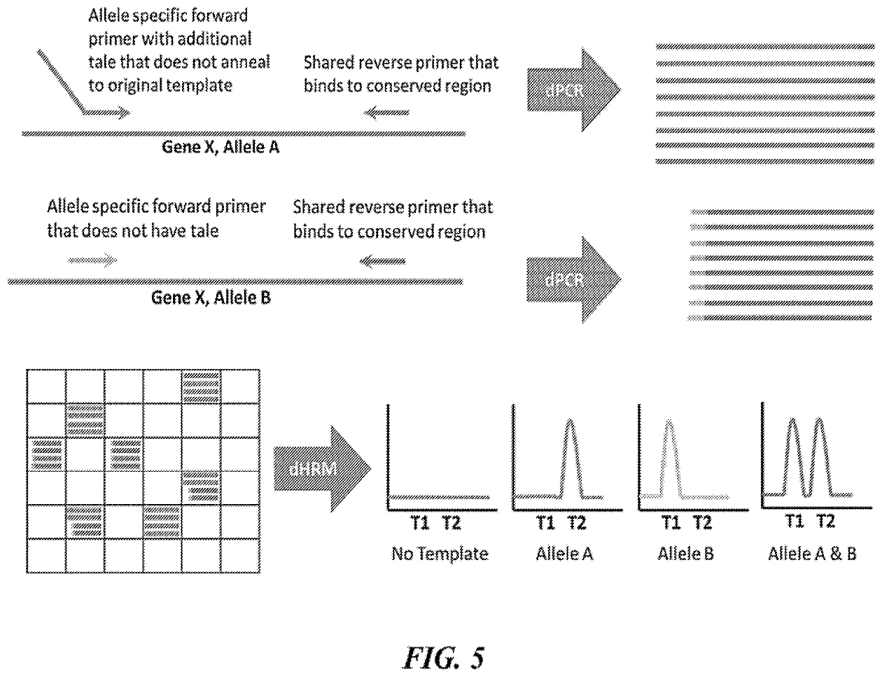

An example method for nucleic acid identification is shown in FIG. 5. The method may include providing a sample comprising one or more target nucleic acid sequences. In an example, the sample may include a first target and a second target nucleic acid sequence. The first and second target nucleic acid sequences may be different alleles, genes, sequences, etc. In an example, the nucleic acid targets are alleles (e.g., allele ‘A’ and allele ‘B’) and the method may include providing, along with the sample, forward primers, reverse primers, and reagents for primer amplification. The forward primers may be allele specific primers. One or more of the forward primers may include a tail or nucleic acid sequence that does not anneal to (e.g., is not complementary to) the nucleic acid sequence for which it is specific. The tail or non-complementary nucleic acid sequence may be located on the 5′ end of the primer. The method may include performing a primer extension reaction to extend the forward primers and amplify the target nucleic acid sequences (e.g., corresponding to allele A and allele B). The forward primers that include the tail or nucleic acid sequence that does not anneal to the target sequence may generate nucleic acid molecules complementary to the target nucleic acid molecule and including the tail sequence. The forward primers without the tail or additional sequence may generate sequences complementary to the target nucleic acid sequence. The extended forward primers may include one or more consensus domains. The method may further include using reverse primers (e.g., universal reverse primers) capable of annealing to the consensus domains to generate copies of the target nucleic acid sequences. A second primer extension reaction may be performed to generate additional amplification products that are copies of the target nucleic acid molecules. For the allele specific forward primers including the tail or added sequences, the copies of the target nucleic acid molecules may also include the tail or added sequence region. The double-stranded copies of the target nucleic acid molecules may be denatured (e.g., via thermal cycling) and, during denaturation signals generated from the separation of the strands of the double-stranded nucleic acid molecules. The denaturation signals may be used to generate denaturation profiles of the target nucleic acid sequences. The presence or absence of a specific denaturation profile may be used to identify a presence or absence or to identify a nucleic acid molecule in the sample. Depending on the sequence differences between the target nucleic acid sequences, the denaturation profiles may at least partially overlap or be difficult to resolve. As such, addition of a tail sequence may shift or alter the denaturation profile of one or more of the target nucleic acid molecules to permit or enhance differences between the denaturation profiles such that the denaturation profiles may be distinguishable from one another.

The double-stranded nucleic acid molecules may be generated in the plurality of chambers. Alternatively, the double-stranded nucleic acid molecules may be generated in a bulk solution and the bulk solution may be partitioned into the plurality of chambers. In an example, the sample is provided to a microfluidic device comprising the plurality of chambers and partitioned into the plurality of chambers. The sample may include at least 1, 2, 3, 4, 5, 6, 8, 10, 15, 20, or more target nucleic acid molecules. In an example, the sample includes at least 10 target nucleic acid molecules. In another example, the sample includes at least 15 target nucleic acid molecules. In another example, the sample includes at least 20 target nucleic acid molecules. In some examples, a target nucleic acid molecule may be a single-stranded or double-stranded nucleic acid molecule. In some cases, a target nucleic acid molecule is circular. A target nucleic acid molecule may comprise one or more modified nucleotides, such as methylated nucleotides and nucleotide analogs. The sample may be diluted such that approximately one nucleic acid molecule is provided to each chamber. The sample may be provided to the chambers with a variety assay reagents and components. For example, the sample may be provided with a plurality of forward primers, reverse primers, and reagents for polymerase chain reaction.

In an example, the sample is provided to the plurality of chambers with a plurality of forward primers. The plurality of forward primers may be universal primers or may be target specific primers. In an example, the plurality of forward primers are not universal primers and are target specific primers. A target specific primer may have sequence complementary with a specific target such that the target specific primer anneals does not anneal to non-target sequences. A forward primer include a single region or may include multiple regions. In an example, the forward primer comprises a single region with sequence complementarity to the target nucleic acid molecule. In another example, the forward primer comprises multiple regions, at least one with sequence complementarity to the target nucleic acid molecule and at least another region, or tail sequence, without sequence complementarity (e.g., non-complementary sequence) to the target nucleic acid molecule. The non-complementarity sequence may not anneal to the target nucleic acid molecule. The tail or non-complementarity sequence may correspond to the added sequence (e.g., sequence added to a double-stranded nucleic acid molecule to alter or shift the denaturation profile). The tail or non-complementary sequence may be a polymeric form of nucleotides of any length. For example, the tail or non-complementary sequence may include at least 1, 2, 3, 4, 5, 6, 8, 10, 15, 20, 30, 40, 50, 100, 500, 1000, or more nucleotides. The added sequence may be added to the double-stranded nucleic acid molecule via one or more of primer extension reactions (e.g., using primer(s) that include the tail or non-complementary sequence) or by ligating a sequence corresponding to the tail or non-complementary sequence to the target nucleic acid molecule or derivative thereof. The nucleotides may include deoxyribonucleotides, ribonucleotides, or analogs thereof. The tail or non-complementary sequence may comprise deoxyribonucleic acid (DNA), ribonucleic acid (RNA), peptide nucleic acid (PNA), lock nucleic acid (LNA), bridge nucleic acid (BNA), or any combination thereof. The tail or non-complementary sequence may include one or more subunits selected from adenosine (A), cytosine (C), guanine (G), thymine (TO, and uracil (U), or variants thereof. A nucleotide can include A, C, G, T, or U, or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (i.e., A or G, or variant thereof) or pyrimidine (i.e., C, T, or U, or variant thereof).

The forward primers may anneal to the target nucleic acid molecules. The annealed forward primers may be subjected to a primer extension reaction. The primer extension reaction may generate extension products of the forward primers (e.g., first extension products) that are complementary to the target nucleic acid molecules. In an example, the forward primers comprise tail sequences or non-complementary sequences and the extension products may further include the tail or non-complementary sequences.

The method may further comprise contacting the extension produces of the forward primers with a plurality of reverse primers. The plurality of reverse primers may include universal primers or target nucleic acid specific primers. In an example, the plurality of reverse primers include universal primers. In another example, the plurality of reverse primers are universal primers and the extension products of the forward primers include consensus sequences complementary to the universal primers. In another example, the plurality of reverse primers are specific to the target nucleic acid molecules. The reverse primers may be annealed to the extension products of the forward primers. The reverse primers may be subjected to a primer extension reaction. The primer extension reaction may generate a plurality of reverse primer extension products (e.g., second extension products). The reverse primer extension products may be the double-stranded nucleic acid molecules that are denatured to generate the denaturation profiles. The reverse primer extension products (e.g., second extension products) may comprise a copy of the target nucleic acid molecule. In an example, the forward primer includes a tail or non-complementary sequence and the copy of the target nucleic acid sequence generated from the reverse primer may include an added sequence complementary to the tail or non-complementary sequence.

The method may comprise providing any microfluidic device as described elsewhere herein. The microfluidic device may comprise at least one channel. The channel may comprise an inlet, an outlet, or both an inlet and an outlet. In an example, the channel comprises a single inlet or port and does not include an outlet or secondary port. In another example, the channel comprises an inlet and an outlet port. The microfluidic device may further comprise a plurality of partitions (e.g., chambers) connected to the channel. The chambers may be connected to the channel by a plurality of siphon apertures. The microfluidic device may be sealed by a thin film (e.g., a thermoplastic thin film) disposed adjacent to a surface of the microfluidic device such that the thin film caps the channel, the plurality of chambers, the plurality of siphon apertures, or any combination thereof. Reagents, the sample, or both may be applied to the inlet of the channel. The fluidic device may be filled by providing a first pressure differential between the reagent or sample and the fluidic device, causing the reagent or sample to flow into the fluidic device. The reagent or sample may be partitioned into the chambers by applying a second pressure differential between the channel and the plurality of chambers to move the reagent or sample into the plurality of chambers and to force gas within the plurality of chambers to pass through the thin film. Alternatively, or in addition to, the fluidic device may include a second channel configured to permit degassing or off-gassing. The second channel may be disposed adjacent to the plurality of chambers. The second pressure differential may be greater than the first pressure differential. A third pressure differential between the inlet and the outlet may be applied to introduce a fluid into the microchannel without introducing the fluid into the chambers. The third pressure differential may be less than the second pressure differential. A reagent may be added before, after, or at the same time as the samples. A reagent may also be provided in one or more partitions of the device by another method. For example, a reagent may be deposited within one or more partitions prior to covering the one or more partitions with the thin film. In an additional example, the plurality of partitions may include reagents dried with the partitions and providing the sample may solubilize the dried reagents.

The inlet or the outlet, if present, of the device may be in fluid communication with a pneumatic pump or a vacuum system. The pneumatic pump or vacuum system may be a component of or separate from a system of the present disclosure. Filling and partitioning of a reagent or sample may be performed by applying pressure differentials across various features of the fluidic device. Filling and partitioning of the reagent or nucleic acid molecules may be performed without the use of valves between the chambers and the channel to isolate reagent or nucleic acid molecules. For example, filling of the channel may be performed by applying a pressure differential between the reagent or sample to be loaded and the channel. This pressure differential may be achieved by pressurizing the reagent or nucleic acid molecules or by applying vacuum to the channel. Filling the chambers may be performed by applying a pressure differential between the channel and the chambers. This may be achieved by pressurizing the channel or by applying a vacuum to the chambers. Partitioning the sample or reagent may be performed by applying a pressure differential between a fluid and the channel. This pressure differential may be achieved by pressurizing the fluid or by applying a vacuum to the channel.

The microfluidic device may include a thin film or second channel (e.g., off-gas channel) that may have different permeability characteristics under different applied pressure differentials. For example, the thin film or second channel may prevent gas flow at the first and third pressure differentials (e.g., low pressure), which may be smaller magnitude pressure differentials. The thin film or second channel may permit gas flow at the second pressure differential (e.g., high pressure), which may be a higher magnitude pressure differential. The first and third pressure differentials may be the same or they may be different. The first pressure differential may be the difference in pressure between the reagent in the inlet or outlet and the microfluidic device. During filling of the microfluidic device, the pressure of the reagent may be higher than the pressure of the microfluidic device. During filling of the fluidic device, the pressure difference between the reagent and the fluidic device (e.g., low pressure) may be less than or equal to about 8 pounds per square inch (psi), less than or equal to about 6 psi, less than or equal to about 4 psi, less than or equal to about 2 psi, less than or equal to about 1 psi, or less. In some examples, during filling of the fluidic device, the pressure differential between the reagent and the microfluidic device may be from about 1 psi to about 8 psi. In some examples, during filling of the fluidic device, the pressure differential between the reagent and the microfluidic device may be from about 1 psi to about 6 psi. In some examples, during filling of the microfluidic device, the pressure differential between the reagent and the microfluidic device may be from about 1 psi to about 4 psi. The fluidic device may be filled by applying a pressure differential between the reagent and the fluidic device for less than or equal to about 20 minutes, less than or equal to about 15 minutes, less than or equal to about 10 minutes, less than or equal to about 5 minutes, less than or equal to about 3 minutes, less than or equal to about 2 minutes, less than or equal about 1 minute, or less.

A filled microfluidic device may have a sample or one or more reagents in the channel, siphon apertures, chambers, or any combination thereof. Backfilling of the sample or the one or more reagents into the chambers may occur upon filling of the fluidic device or may occur during application of a second pressure differential. The second pressure differential (e.g., high pressure) may correspond to the difference in pressure between the channel and the plurality of chambers. During application of the second pressure differential a first fluid (e.g., gas or liquid) in the higher pressure domain may push a second fluid (e.g., gas) in the lower pressure domain through the thin film and out of the fluidic device. The first and second fluids may comprise a liquid or a gas. The liquid may comprise an aqueous mixture or an oil mixture. The second pressure differential may be achieved by pressurizing the channel. Alternatively, or in addition, the second pressure differentially may be achieved by applying a vacuum to the chambers. During application of the second pressure differential, nucleic acid molecules or reagents in the channel may flow into the chambers. Additionally, during the application of the second pressure differential gas trapped within the siphon apertures, chambers, and channel may outgas through the thin film or through one or more walls of the chambers and into a second channel (e.g., off-gas channel). During backfilling and outgassing of the chambers, the pressure differential between the chambers and the channel may be greater than or equal to about 6 psi, greater than or equal to about 8 psi, greater than or equal to about 10 psi, greater than or equal to about 12 psi, greater than or equal to about 14 psi, greater than or equal to about 16 psi, greater than or equal to about 18 psi, greater than or equal to about 20 psi, or greater. In some examples, during backfilling of the chambers, the pressure differential between the chambers and the channel is from about 8 psi to about 20 psi. In some examples, during backfilling of the chambers, the pressure differential between the chambers and the channel is from about 8 psi to about 18 psi. In some examples, during backfilling of the chambers, the pressure differential between the chambers and the channel is from about 8 psi to about 16 psi. In some examples, during backfilling of the chambers, the pressure differential between the chambers and the microchannel is from about 8 psi to about 14 psi. In some examples, during backfilling of the chambers, the pressure differential between the chambers and the channel is from about 8 psi to about 12 psi. In some examples, during backfilling of the chambers, the pressure differential between the chambers and the channel is from about 8 psi to about 10 psi. The chambers may be backfilled and outgassed by applying a pressure differential for more than about 5 minutes, more than about 10 minutes, more than about 15 minutes, more than about 20 minutes, more than about 25 minutes, more than about 30 minutes, or more.

The plurality of chambers may have greater than or equal to about 1,000 chambers, 5,000 chambers, 10,000 chambers, 20,000 chambers, 30,000 chambers, 40,000 chambers, 50,000 chambers, 100,000 chambers, or more. In an example, the microfluidic device may have from about 10,000 to 30,000 chambers. In another example, the microfluidic device may have from about 15,000 to 25,000 chambers. In an example, the plurality of chambers comprises greater than or equal to about 1,000 chambers. In another example, the plurality of chambers comprises greater than or equal to about 10,000 chambers. The chambers may be cylindrical in shape, hemispherical in shape, or a combination of cylindrical and hemispherical in shape. Alternatively, or in addition to, the chambers may be cubic in shape. The chambers may have a cross-sectional dimension of less than or equal to about 500 μm, 250 μm, 100 μm, 80 μm, 60 μm, 30 μm, 15 μm, or less. In an example, the chamber has a cross-sectional dimension (e.g., diameter or side length) that is less than or equal to about 250 μm. In another example, the chamber has a cross-sectional dimension (e.g., diameter or side length) that is less than or equal to about 100 μm. In another example, the chamber has a cross-sectional dimension (e.g., diameter or side length) that is less than or equal to about 50 μm. The chambers may have any volume. The chambers may have the same volume or the volume may vary across the microfluidic device. The chambers may have a volume of less than or equal to about 1000 picoliters (pL), 900 pL, 800 pL, 700 pL, 600 pL, 500 pL, 400 pL, 300 pL, 200 pL, 100 pL, 75 pL, 50 pL, 25 pL, or less picoliters. The chambers may have a volume from about 25 pL to 50 pL, 25 pL to 75 pL, 25 pL to 100 pL, 25 pL to 200 pL, 25 pL to 300 pL, 25 pL to 400 pL, 25 pL to 500 pL, 25 pL to 600 pL, 25 pL to 700 pL, 25 pL to 800 pL, 25 pL to 900 pL, or 25 pL to 1000 pL. In an example, the chamber(s) have a volume of less than or equal to 500 pL. In another example, the chambers have a volume of less than or equal to about 250 pL. In another example, the chambers have a volume of less than or equal to about 100 pL.

Partitioning of the sample may be verified by the presence of an indicator within the reagent. An indicator may include a molecule comprising a detectable moiety. The detectable moiety may include radioactive species, fluorescent labels, chemiluminescent labels, enzymatic labels, colorimetric labels, or any combination thereof. Non-limiting examples of radioactive species include ³H, ¹⁴C, ²²Na, ³²P, ³³P, ³⁵S, ⁴²K, ⁴⁵Ca, ⁵⁹Fe, ¹²³I, ¹²⁴I, ¹²⁵I, ¹³¹I, or ²⁰³Hg. Non-limiting examples of fluorescent labels include fluorescent proteins, optically active dyes (e.g., a fluorescent dye), organometallic fluorophores, or any combination thereof. Non-limiting examples of chemiluminescent labels include enzymes of the luciferase class such as Cypridina, Gaussia, Renilla, and Firefly luciferases. Non-limiting examples of enzymatic labels include horseradish peroxidase (HRP), alkaline phosphatase (AP), beta galactosidase, glucose oxidase, or other labels.

An indicator molecule may be a fluorescent molecule. Fluorescent molecules may include fluorescent proteins, fluorescent dyes, and organometallic fluorophores. The indicator molecule may be a protein fluorophore. Protein fluorophores may include green fluorescent proteins (GFPs, fluorescent proteins that fluoresce in the green region of the spectrum, generally emitting light having a wavelength from 500-550 nanometers), cyan-fluorescent proteins (CFPs, fluorescent proteins that fluoresce in the cyan region of the spectrum, generally emitting light having a wavelength from 450-500 nanometers), red fluorescent proteins (RFPs, fluorescent proteins that fluoresce in the red region of the spectrum, generally emitting light having a wavelength from 600-650 nanometers). Non-limiting examples of protein fluorophores include mutants and spectral variants of AcGFP, AcGFP1, AmCyan, AmCyan1, AQ143, AsRed2, Azami Green, Azurite, BFP, Cerulean, CFP, CGFP, Citrine, copGFP, CyPet, dKeima-Tandem, DsRed, dsRed-Express, DsRed-Monomer, DsRed2, dTomato, dTomato-Tandem, EBFP, EBFP2, ECFP, EGFP, Emerald, EosFP, EYFP, GFP, HcRed-Tandem, HcRed1, JRed, Katuska, Kusabira Orange, Kusabira Orange2, mApple, mBanana, mCerulean, mCFP, mCherry, mCitrine, mECFP, mEmerald, mGrape1, mGrape2, mHoneydew, Midori-Ishi Cyan, mKeima, mKO, mOrange, mOrange2, mPlum, mRaspberry, mRFP1, mRuby, mStrawberry, mTagBFP, mTangerine, mTeal, mTomato, mTurquoise, mWasabi, PhiYFP, ReAsH, Sapphire, Superfolder GFP, T-Sapphire, TagCFP, TagGFP, TagRFP, TagRFP-T, TagYFP, tdTomato, Topaz, TurboGFP, Venus, YFP, YPet, ZsGreen, and ZsYellow1.

The indicator molecule may be a fluorescent dye. Non-limiting examples of fluorescent dyes include SYBR green; SYBR blue; DAPI; propidium iodine; Hoeste; SYBR gold; ethidium bromide; acridines; proflavine; acridine orange; acriflavine; fluorcoumanin; ellipticine; daunomycin; chloroquine; distamycin D; chromomycin; homidium; mithramycin; ruthenium polypyridyls; anthramycin; phenanthridines and acridines; propidium iodide; hexidium iodide; dihydroethidium; ethidium monoazide; ACMA; Hoechst 33258; Hoechst 33342; Hoechst 34580; DAPI; acridine orange; 7-AAD; actinomycin D; LDS751; hydroxystilbamidine; SYTOX Blue; SYTOX Green; SYTOX Orange; POPO-1; POPO-3; YOYO-1; YOYO-3; TOTO-1; TOTO-3; JOJO-1; LOLO-1; BOBO-1; BOBO-3; PO-PRO-1; PO-PRO-3; BO-PRO-1; BO-PRO-3; TO-PRO-1; TO-PRO-3; TO-PRO-5; JO-PRO-1; LO-PRO-1; YO-PRO-1; YO-PRO-3; PicoGreen; OliGreen; RiboGreen; SYBR Gold; SYBR Green I; SYBR Green II; SYBR DX; SYTO-40, SYTO-41, SYTO-42, SYTO-43, SYTO-44, and SYTO-45 (blue); SYTO-13, SYTO-16, SYTO-24, SYTO-21, SYTO-23, SYTO-12, SYTO-11, SYTO-20, SYTO-22, SYTO-15, SYTO-14, and SYTO-25 (green); SYTO-81, SYTO-80, SYTO-82, SYTO-83, SYTO-84, and SYTO-85 (orange); SYTO-64, SYTO-17, SYTO-59, SYTO-61, SYTO-62, SYTO-60, and SYTO-63 (red); fluorescein; fluorescein isothiocyanate (FITC); tetramethyl rhodamine isothiocyanate (TRITC); rhodamine; tetramethyl rhodamine; R-phycoerythrin; Cy-2; Cy-3; Cy-3.5; Cy-5; Cy5.5; Cy-7; Texas Red; Phar-Red; allophycocyanin (APC); Sybr Green I; Sybr Green II; Sybr Gold; CellTracker Green; 7-AAD; ethidium homodimer I; ethidium homodimer II; ethidium homodimer III; umbelliferone; eosin; green fluorescent protein; erythrosin; coumarin; methyl coumarin; pyrene; malachite green; stilbene; lucifer yellow; cascade blue; dichlorotriazinylamine fluorescein; dansyl chloride; fluorescent lanthanide complexes such as those including europium and terbium; carboxy tetrachloro fluorescein; 5 or 6-carboxy fluorescein (FAM); 5- (or 6-) iodoacetamidofluorescein; 5-{[2(and 3)-5-(Acetylmercapto)-succinyl]amino}fluorescein (SAMSA-fluorescein); lissamine rhodamine B sulfonyl chloride; 5 or 6 carboxy rhodamine (ROX); 7-amino-methyl-coumarin; 7-Amino-4-methylcoumarin-3-acetic acid (AMCA); BODIPY fluorophores; 8-methoxypyrene-1;3;6-trisulfonic acid trisodium salt; 3;6-Disulfonate-4-amino-naphthalimide; phycobiliproteins; AlexaFluor 350, 405, 430, 488, 532, 546, 555, 568, 594, 610, 633, 635, 647, 660, 680, 700, 750, and 790 dyes; DyLight 350, 405, 488, 550, 594, 633, 650, 680, 755, and 800 dyes; and other fluorophores.

The indicator molecule may be an organometallic fluorophore. Non-limiting examples of organometallic fluorophores include lanthanide ion chelates, non-limiting examples of which include tris(dibenzoylmethane) mono(1,10-phenanthroline)europium(lll), tris(dibenzoylmethane) mono(5-amino-1,10-phenanthroline)europium (lll), and Lumi4-Tb cryptate.

The microfluidic device may be filled with one or more amplification reagents such as nucleic acid molecules, components for an amplification reaction (e.g., primers, polymerases, and deoxyribonucleotides), an indicator molecule, and an amplification probe. Amplification reactions may involve thermal cycling the plurality of microchambers or a subset thereof, as described herein. Detection of nucleic acid amplification may be performed by collecting signals from (e.g., imaging) the plurality of chambers of the microfluidic device or a subset thereof. Nucleic acid molecules may be quantified by counting the microchambers in which the nucleic acid molecules are successfully amplified and applying Poisson statistics. The nucleic acid molecule may be partitioned such that a partition comprises one or less nucleic acid molecule. Alternatively, or in addition to, a partition may include multiple nucleic acid molecules. Nucleic acid molecules may also be quantified by processing signals collected at different time points throughout an amplification reaction. For example, one or more signals may be collected during each thermal cycle (e.g., each amplification cycle) of a nucleic acid amplification reaction and the signals can be used to determine an amplification rate as in, e.g., real-time or quantitative polymerase chain reaction (real-time PCR or qPCR). Nucleic acid amplification and quantification may be performed in a single integrated unit, e.g., within a given partition or a subset of the plurality of partitions of the device. In some examples, nucleic acid amplification may be detected and monitored in real-time.

A variety of nucleic acid amplification reactions may be used to amplify the nucleic acid molecule in a sample to generate an amplified product. Amplification of a nucleic acid target may be linear, exponential, or a combination thereof. Non-limiting examples of nucleic acid amplification methods include primer extension, polymerase chain reaction, reverse transcription, isothermal amplification, ligase chain reaction, helicase-dependent amplification, asymmetric amplification, rolling circle amplification, and multiple displacement amplification. The amplification product of an amplification reaction may be DNA or RNA. For samples including DNA molecules, any DNA amplification method may be employed. DNA amplification methods include, but are not limited to, PCR, real-time PCR, assembly PCR, asymmetric PCR, digital PCR, dial-out PCR, helicase-dependent PCR, nested PCR, hot start PCR, inverse PCR, methylation-specific PCR, miniprimer PCR, multiplex PCR, overlap-extension PCR, thermal asymmetric interlaced PCR, touchdown PCR, and ligase chain reaction. DNA amplification may be linear, exponential, or any combination thereof. DNA amplification may also be achieved with digital PCR (dPCR), real-time quantitative PCR (qPCR), or quantitative digital PCR (qdPCR), as described herein.

Reagents used for nucleic acid amplification may include polymerizing enzymes, reverse primers, forward primers, and amplification probes. Examples of polymerizing enzymes include, without limitation, nucleic acid polymerase, transcriptase, or ligase (i.e., enzymes which catalyze the formation of a bond). The polymerizing enzyme can be naturally occurring or synthesized. Examples of polymerases include a DNA polymerase, and RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase Pwo polymerase, VENT polymerase, DEEPVENT polymerase, Ex-Taq polymerase, LA-Taw polymerase, Sso polymerase Poc polymerase, Pab polymerase, Mth polymerase ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Pfutubo polymerase, Pyrobest polymerase, KOD polymerase, Bst polymerase, Sac polymerase, Klenow fragment polymerase with 3′ to 5′ exonuclease activity, and variants, modified products and derivatives thereof. For a Hot Start polymerase, denaturation at a temperature from about 92° C. to 95° C. for a time period from about 2 minutes to 10 minutes may be used.

A nucleic acid amplification reaction may involve an amplification probe. An amplification probe may be a sequence-specific oligonucleotide probe. The amplification probe may be optically active when hybridized with an amplification product. The amplification probe may be detectable as nucleic acid amplification progresses. The intensity of a signal collected from a plurality of partitions including nucleic acid molecules (e.g., optical signal) may be proportional to the amount of amplified product included in the partitions. For example, the signal collected from a particular partition may be proportional to the amount of amplified product in that particular partition. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, Lion probes, locked nucleic acid probes, or molecular beacons. Non-limiting examples of quenchers that may be useful in blocking the optical activity of the probe include Black Hole Quenchers (BHQ), Iowa Black FQ and RQ quenchers, or Internal ZEN Quenchers. Alternatively, or in addition, the probe or quencher may be any probe that is useful in the context of the methods of the present disclosure.

The amplification probe may be a dual labeled fluorescent probe. The dual labeled probe may include a fluorescent reporter and a fluorescent quencher linked with a nucleic acid. The fluorescent reporter and fluorescent quencher may be positioned in close proximity to each other. The close proximity of the fluorescent reporter and fluorescent quencher may block the optical activity of the fluorescent reporter. The dual labeled probe may bind to the nucleic acid molecule to be amplified. During amplification, the fluorescent reporter and fluorescent quencher may be cleaved by the exonuclease activity of the polymerase. Cleaving the fluorescent reporter and quencher from the amplification probe may cause the fluorescent reporter to regain its optical activity and enable detection. The dual labeled fluorescent probe may include a 5′ fluorescent reporter with an excitation wavelength maximum of about 450 nanometers (nm), 500 nm, 525 nm, 550 nm, 575 nm, 600 nm, 625 nm, 650 nm, 675 nm, 700 nm, or higher and an emission wavelength maximum of about 500 nm, 525 nm, 550 nm, 575 nm, 600 nm, 625 nm, 650 nm, 675 nm, 700 nm, or higher. The dual labeled fluorescent probe may also include a 3′ fluorescent quencher. The fluorescent quencher may quench fluorescent emission wavelengths between about 380 nm and 550 nm, 390 nm and 625 nm, 470 nm and 560 nm, 480 nm and 580 nm, 550 nm and 650 nm, 550 nm and 750 nm, or 620 nm and 730 nm.

Nucleic acid amplification may include multiple cycles of thermal cycling (e.g., multiple amplification cycles). Any suitable number of cycles may be performed. The number of cycles performed may be more than about 5, more than about 10, more than about 15, more than about 20, more than about 30, more than about 40, more than about 50, more than about 60, more than about 70, more than about 80, more than about 90, more than about 100 cycles, or more. The number of cycles performed may depend upon the number of cycles to obtain detectable amplification products. For example, the number of cycles to detect nucleic acid amplification during PCR (e.g., dPCR, qPCR, or qdPCR) may be less than or equal to about 100, less than or equal to about 90, less than or equal to about 80, less than or equal to about 70, less than or equal to about 60, less than or equal to about 50, less than or equal to about 40, less than or equal to about 30, less than or equal to about 20, less than or equal to about 15, less than or equal to about 10, less than or equal to about 5 cycles, or less. Nucleic acid amplification may be monitored in real-time. In an example, nucleic acid amplification is monitored as a function of the number of cycles performed. Monitoring nucleic acid amplification may permit detection of false positives. For example, a partition that does not follow an expected amplification trend may generate a false positive. The false positive may be excluded from additional analysis. Monitoring nucleic acid amplification as a function of number of amplification cycles may permit a reduction in the amount of data collected and increase in analysis speed. The nucleic acid amplification reaction may be monitored (e.g., signal may be collected) every 2, 4, 6, 8, 10, 12, 15, 20, or more cycles. In an example, amplification signals are collected at least every other cycle. In another example, amplification signals are collected at least every 4 cycles. In another example, amplification signals are collected at least every 10 cycles.

The method may further comprise denaturing the double-stranded nucleic acid molecules. The double-stranded nucleic acid molecule may be denatured using thermal energy, acids or bases (e.g., sodium hydroxide treatment), organic solvents, salts, or any combination thereof. Denaturation of the double-stranded nucleic acid molecules may be reversible (e.g., via thermal denaturation) or irreversible (e.g., via salts or organic solvents). In an example, the double-stranded nucleic acid molecules are denatured by thermal energy. Denaturation temperatures may vary depending upon, for example, the nucleic acid molecule, the reagents used, and the reaction conditions.

The double-stranded nucleic acid molecule may be thermally denatured via controlled heating of the nucleic acid molecule or derivative thereof. In an example, the double-stranded nucleic acid molecule(s) are disposed in a plurality of partitions and the partitions undergo controlled heating. Controlled heating may include resistive heating, radiative heating, conductive heating, convective heating, thermoelectric heating, or any combination thereof. Thermal denaturation may include controlled heating of the double-stranded nucleic acid molecule to a temperature of from about 60° C. to 70° C., 60° C. to 80° C., 60° C. to 90° C., 60° C. to 100° C., 60° C. to 110° C., 70° C. to 80° C., 70° C. to 90° C., 70° C. to 100° C., 70° C. to 110° C., 80° C. to 90° C., 80° C. to 100° C., 80° C. to 110° C., 90° C. to 100° C., 90° C. to 110° C., or 100° C. to 110° C. for a given period of time. In an example, the double-stranded nucleic acid molecule may undergo controlled heating from about 60° C. to about 90° C. for a given time. In another example, the double-stranded nucleic acid molecule may undergo controlled heating from about 65° C. to about 85° C. for a given time. Thermal denaturation may include controlled heating of the double-stranded nucleic acid molecule to a temperature of greater than or equal to about 60° C., 65° C., 70° C., 80° C., 85° C., 90° C., 95° C., or higher for a given period of time.

The duration of thermal denaturation may vary depending upon, for example, the particular nucleic acid molecule, the reagents used, and the reaction conditions. The duration of thermal denaturation may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. Alternatively, the duration for denaturation may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Controlled heating of a subset of the plurality of partitions of a device may be performed at any useful rate and over any useful temperature range. For example, controlled heating may be performed from a lower temperature of at least about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C., or more. Controlled heating may be performed to an upper temperature of at least about 35° C., about 40° C., about 45° C., about 50° C., about 55° C., about 60° C., about 65° C., about 70° C., about 75° C., about 80° C., about 85° C., about 90° C., about 91° C., about 92° C., about 93° C., about 94° C., about 95° C., about 96° C., about 97° C., about 98° C., about 99° C., or about 100° C., or more. Temperature may be increased by any useful increment. For example, temperature may be increased by at least about 0.01° C., about 0.05° C., about 0.1° C., about 0.2° C., about 0.3° C., about 0.4° C., about 0.5° C., about 1° C., about 2° C., about 3° C., about 4° C., about 5° C., or about 10° C., or more. Controlled heating may also occur over evenly or evenly spaced temperature increments. For example, temperature may be increased by about 0.1° C. over a range where significant melting of a nucleic acid molecule is expected (e.g., fine grain measurement) and by about 1° C. over a range where no significant melting of a nucleic acid molecule is expected (e.g., coarse grain measurement). Controlled heating may be performed at any useful rate such as at least about 0.0001° C./second, about 0.0002° C./second, about 0.0003° C./second, about 0.0004° C./second, about 0.0005° C./second, about 0.0006° C./second, about 0.0007° C./second, about 0.0008° C./second, about 0.0009° C./second, about 0.001° C./second, about 0.002° C./second, about 0.003° C./second, about 0.004° C./second, about 0.005° C./second, about 0.006° C./second, about 0.007° C./second, about 0.008° C./second, about 0.009° C./second, about 0.01° C./second, about 0.02° C./second, about 0.03° C./second, about 0.04° C./second, about 0.05° C./second, about 0.06° C./second, about 0.07° C./second, about 0.08° C./second, about 0.09° C./second, about 0.1° C./second, about 0.2° C./second, about 0.3° C./second, about 0.4° C./second, about 0.5° C./second, about 0.6° C./second, about 0.7° C./second, about 0.8° C./second, about 0.9° C./second, about 1° C./second, about 2° C./second, about 3° C./second, about 4° C./second, and about 5° C./second, or more. A thermal unit (e.g., a heater) carrying out the controlled heating process may maintain a given temperature for any useful duration. For example, a given temperature may be maintained for at least about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 45 seconds, about 60 seconds, about 70 seconds, about 80 seconds, about 90 seconds, about 100 seconds, about 110 seconds, about 120 seconds, about 130 seconds, about 140 seconds, about 150 seconds, about 160 seconds, about 170 seconds, about 180 seconds, about 190 seconds, about 200 seconds, about 210 seconds, about 220 seconds, about 230 seconds, about 240 seconds, about 250 seconds or about 300 seconds, or more.

The method may further include collecting signals during denaturation of the double-stranded nucleic acid molecules. Signals may include optical signals, electrical signals, or any combination thereof. In an example, signals collected are optical signals. Collection of optical signals may include imaging partitions of the microfluidic device or a portion of the partitions of the microfluidic device. Signals may be collected from the subset of the plurality of partitions at any selected time points. For example, signal may be collected at least about every 1 second, about every 2 seconds, about every 3 seconds, about every 4 seconds, about every 5 seconds, about every 6 seconds, about every 7 seconds, about every 8 seconds, about every 9 seconds, about every 10 seconds, about every 20 seconds, about every 30 seconds, about every 45 seconds, about every 60 seconds, about every 70 seconds, about every 80 seconds, about every 90 seconds, about every 100 seconds, about every 110 seconds, about every 120 seconds, about every 130 seconds, about every 140 seconds, about every 150 seconds, about every 160 seconds, about every 170 seconds, about every 180 seconds, about every 190 seconds, about every 200 seconds, about every 210 seconds, about every 220 seconds, about every 230 seconds, about every 240 seconds, about every 250 seconds, or about every 300 seconds, or more. Alternatively, or in addition to, signals may be collected at select temperature intervals. For example, signals may be collected at temperature intervals of less than or equal to about 5° C., 4° C., 3° C., 2.5° C., 2° C., 1.5° C., 1° C., 0.5° C., 0.25° C., or less. Signals may be collected (e.g., images taken) from the microfluidic device or a subset of the plurality of partitions (e.g., microchambers) thereof. Collecting signals may comprise taking images of the device or a subset of the plurality of partitions thereof. Signals (e.g., images) may be collected from single microchambers, an array of microchambers, or of multiple arrays of microchambers concurrently. Signals may be collected through the body of the microfluidic device, through the thin film of the microfluidic device, or both. The body of the microfluidic device may be substantially optically transparent. Alternatively, the body of the microfluidic device may be substantially optically opaque. Similarly, the thin film may be substantially optically transparent. Alternatively, the body of the microfluidic device may be substantially optically opaque.

The method may further include using intercalating dyes. The intercalating dyes may generate the detectable signals. The method may include the use of a single type of intercalating dye (e.g., fluorescent dye with a first emission wavelength) or the method may include the use of multiple types of intercalating dyes (e.g., multiple fluorescence dyes with multiple emission wavelengths). The detectable signals produced by the method may be presented or read as raw fluorescence units, relative fluorescence units, copy numbers (cp), copy numbers in relation to volume (e.g., cp/μL), critical threshold of amplification (Ct), amplification cycles, concentration, absolute numbers, derivative reporter, arbitrary units, or any combination thereof. Intercalating dyes may be inserted into the nucleic acid molecule during amplification of the nucleic acid molecule. Intercalating dyes may non-specifically interact or bind to double-stranded nucleic acid molecules. Association of the intercalating dye with a double-stranded nucleic acid molecule may permit detectable fluorescence of the intercalating dye. Denaturation of the nucleic acid molecule may quench or otherwise extinguish the fluorescence signal from the intercalating dye. Non-limiting examples of intercalating dyes include SYBR green, EvaGreen, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines, LCGReen, or any combination thereof.

The method may further include processing the collected signals to generate denaturation profiles of the nucleic acid molecules. Alternatively, or in addition to, the collected signals may be processed to generate denaturation profiles for an individual partition comprising one or more double-stranded nucleic acid molecules. Processing collected signals may comprise using the signals to generate denaturation curves (e.g., denaturation profiles), such as signal versus temperature curves or signal versus concentration curves (e.g., for base, salt, or organic solvent denaturation). Signal may include optical signal (e.g., fluorescence intensity) or non-optical signals (e.g., electrical signals). Processing the signals may further include plotting the negative first derivative of the denaturation curve to determine the denaturation temperature or denaturant concentration. Processing the signals may further include performing melt curve analysis to determine a denaturation profile or dissociation characteristics (e.g., melting temperature) of the nucleic acid molecule or plurality of nucleic acid molecules. The intercalating dye may generate a signal when associated with a double-stranded nucleic acid molecule. As the double-stranded nucleic acid molecule is denatured and forms random coils, the intercalating dye may disassociate from the single stranded nucleic acid molecules and the signal may decrease or go to zero.

A double-stranded nucleic acid molecule may have a different denaturation profile from another double-stranded nucleic acid molecule. For example, a first double-stranded nucleic acid molecule may generate a first denaturation profile and a second double-stranded nucleic acid molecule may generate a second denaturation profile. The first denaturation profile may permit detection, identification, or quantification of a first target nucleic acid molecule and the second denaturation profile may permit detection, identification, or quantification of a second target nucleic acid molecule. In some cases, processing the collected signals may include generating 1, 2, 3, 4, 5, 6, 7, 8, 9, 10, 11, 12, or 13 denaturation profiles for 1, 2, 3, 4, 5, 6, 7, 8, 9, 10, 11, 12, or 13 double-stranded nucleic acid molecules that correspond to 1, 2, 3, 4, 5, 6, 7, 8, 9, 10, 11, 12, or 13 different target nucleic acid molecules. In some cases, the target nucleic acid molecules are provided in a single sample or multiple samples. A denaturation profile of a double-stranded nucleic acid molecule may be dependent upon nucleobases that make up the nucleic acid molecule, length (e.g., number of bases) of the nucleic acid molecule, type of nucleotides that make up the nucleic acid molecule (e.g., PNA, DNA, BNA, LNA, etc.), concentration of nucleic acid molecules, or any combination thereof. A double-stranded nucleic acid molecule may be different from another double-stranded nucleic acid molecule. A denaturation profile of a first double-stranded nucleic acid molecule may be modulated or shifted using the forward primer with the tail or non-complementary sequence. The tail or non-complementary sequence may add additional nucleobases to the double-stranded nucleic acid that may alter the denaturation profile of the double-stranded nucleic acid molecule via altering the length or nucleobases present in the tail or non-complementary sequence. A difference between the double-stranded nucleic acid molecule and another double-stranded nucleic acid molecule may permit the double-stranded nucleic acid molecule to be distinguished via a denaturation profile from another double-stranded nucleic acid molecule. A characteristic (e.g., melting temperature, concentration of denaturation, etc.) of the denaturation profile of the double-stranded nucleic acid molecule may be at least about 0.5%, 1%, 2%, 3%, 4%, 5%, 6%, 8%, 10%, 12%, 15%, 20%, 30%, 40%, or more different from a denaturation profile of another double-stranded nucleic acid molecule. For example, a melting temperature of the double-stranded nucleic acid molecule may be at least about 0.5% different from a melting temperature of another double-stranded nucleic acid molecule. In another example, a melting temperature of the double-stranded nucleic acid molecule may be at least about 1% different from a melting temperature of another double-stranded nucleic acid molecule. In another example, a melting temperature of the double-stranded nucleic acid molecule may be at least about 2% different from a melting temperature of another double-stranded nucleic acid molecule. In another example, a melting temperature of the double-stranded nucleic acid molecule may be at least about 5% different from a melting temperature of another double-stranded nucleic acid molecule.

In an example, a denaturation profile of a double-stranded nucleic acid molecule includes a melting point of the double-stranded nucleic acid molecule and is different from a denaturation profile (e.g., melting point) of another double-stranded nucleic acid molecule. The melting point of a double-stranded nucleic acid molecule may be at least about 0.1° C., 0.2° C., 0.3° C., 0.4° C., 0.5° C., 0.6° C., 0.8° C., 1° C., 1.5° C., 2° C., 3° C., 4° C., 5° C., 6° C., 8° C., 10° C., or more degrees different than a melting point of another double-stranded nucleic acid molecule. In an example, the melting point of a double-stranded nucleic acid molecule may be at least 0.25° C. different than a melting point of another double-stranded nucleic acid molecule. In another example, the melting point of a double-stranded nucleic acid may be at least 0.5° C. different than a melting point of another double-stranded nucleic acid molecule. The melting point may be derived from a first derivative plot of the nucleic acid molecule. Alternatively, or in addition, melting curve subtraction may be used to distinguish a melting point of a nucleic acid molecule from another nucleic acid molecule and, thus, distinguish identity of one analyte from another.

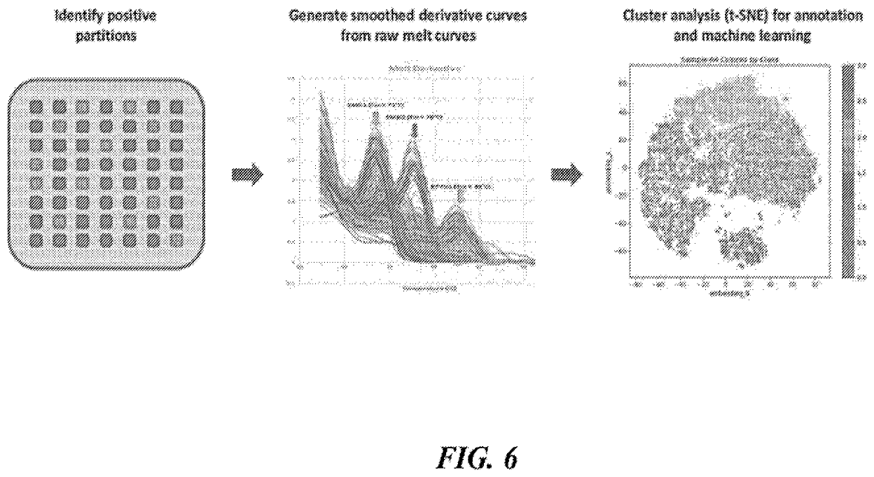

The methods and systems described herein may be evaluated using a variety of assays. In an example, a spinal muscular atrophy (SMA) assay may be used to evaluate the methods and integrated system. FIG. 6 shows an example software workflow for verifying instrument performance. In this example, during melt curve analysis, temperature may be ramped at 0.05° C. per second between 60° C. to 90° C. At each temperature step, an image containing all digital reaction chambers may be acquired. The raw melt curves may be smoothened by a Savitzky-Golay smoothing filter with user-defined parameters, followed by interpolation with temperature adjustment such that the melting temperature (Tₘ) of an internal calibrator being aligned across profiles obtained from all chambers. After calibration of chamber-to-chamber variations (e.g., for both optical signals and temperature) and curve smoothing, melt curves may be normalized to decouple the contribution of the temperature-dependent background fluorescence change from the true fluorescent signals associated with DNA melting. The integrated system may be assessed on a variety of parameters include detection sensitivity, quantification, and reproducibility based on limit of blank detection, limit of detection, and limit of quantitation. Limit of blank detection may be determined using either no-template controls or PCR reagent without DNA templates and may be determined as averaged signals observed from the blank samples. The limit of detection may be determined using genomic copy numbers for serially diluted DNA samples. The lowest reliable quantitated number of genomic copies may depend on the standard deviation measured in the replicates. The limit of quantitation may be determined via quantification of 0.001/partition to 1/partitions (e.g., four decades) of genomic DNA.

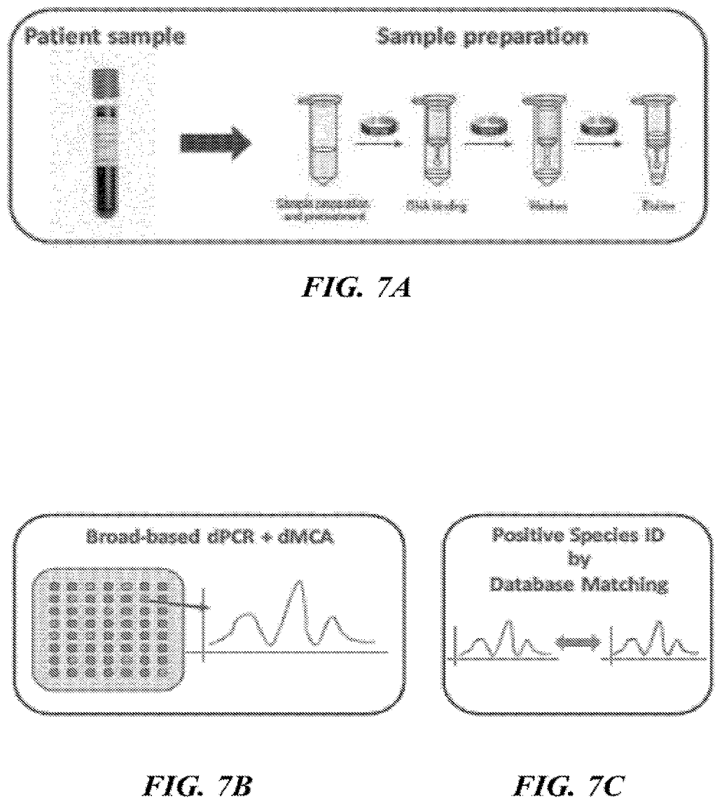

The methods described herein may be used for a variety of experimental processes. For example, method and system performance may be demonstrated using a purified DNA sample. The DNA sample may be assessed to determine crude DNA concentration of the sample. The DNA sample may be combined with a mastermix assay, reagents for a broad-based PCR assay, and intercalating dyes. FIGS. 7A-7C shows an example process for demonstrating system performance. FIG. 7A shows an example sample preparation workflow to quantify and purify DNA within the sample. Sample partitioning, target amplification, and denaturation may be performed as described elsewhere herein. Example partitioning and denaturation profiles are shown in FIG. 7B. For melt curve matching, calibrated, normalized melt curves may be acquired from the software and passed into a database of melt curves from known samples to match and identify the unknown sample, as shown in FIG. 7C. A classifier (e.g., Naïve-Bayes classifier) may be used to output a posterior probability for each possible class. In this example, the classes may be known organism labels corresponding to their respective melt curves, and the posterior probability may be the probability of the sample belonging to that class. The classifier may apply Bayes theorem and is the product of the prior probabilities of the classes P(C) and a likelihood function, which may be the probability of observations for a given class P(X|C). Here the likelihood function can be described as a measure of similarity between melt curves. To account more for similarities in shape, the model may use a Hilbert transformation to convolve the curves. The curves may then be compared the original values as well as the complex values from the transform for the sample and class curves to determine the distance between them. The highest posterior probability may indicate that the sample is most likely that organism.

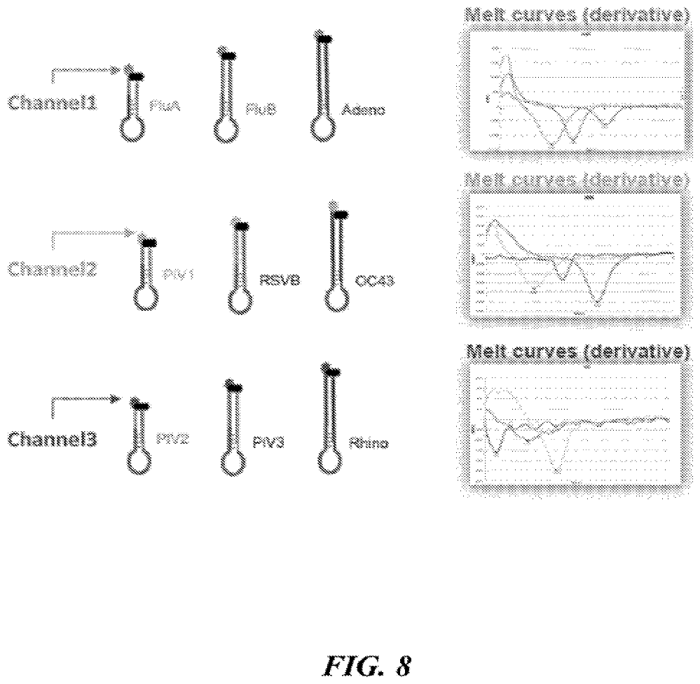

The methods and integrated platform described herein may be used for probe-based denaturation curve generation. Probe-based denaturation curves may be used to improve multiplexity. FIG. 8 shows an example where the probe-based technology is used for panel-based respiratory pathogen detection. Probe-based multiplexing may be used to determine melt temperature, however, may not be able to quantify targets accurately as multiplexing in a bulk reaction may affect the PCR amplification efficiency among the targets, making it extremely challenging to correlate with standard curves. By running the assays on an integrated dPCR platform, it may enable multiplexed quantification using melt curves. Furthermore, the probe-based denaturation curve generation integrated with the dPCR platform may provide for cost-effective, sensitive, multi-gene patient longitudinal monitoring applications which may impact infectious disease and oncology patient care.

### Systems and Devices for Nucleic Acid Identification

In another aspect, the present disclosure provides systems for nucleic acid identification. The system may include a detection unit and one or more processors. The detection unit may be configured to collect or may collect and process signals for identification of nucleic acid molecules. The one or more processors may be operatively coupled to the detection unit and may be individually or collectively programed or otherwise configured to execute the methods described elsewhere herein. For example, the processors may be programed or configured to generate, in a plurality of chambers, a plurality of double-stranded nucleic acid molecules from a plurality of nucleic acid molecules, denature the double-stranded nucleic acid molecules, detect signals indicative of the denaturation of the double-stranded nucleic acid molecules to generate a plurality of denaturation profiles, and process the denaturation profiles to identify at least one nucleic acid molecule. The plurality of double-stranded nucleic acid molecules may include a first subset of double-stranded nucleic acid molecules and a second subset of double-stranded nucleic acid molecules. The first subset of double-stranded nucleic acid molecules may include a first double-stranded nucleic acid molecule comprising a first sequence corresponding to a first nucleic acid molecule and an added sequence. The plurality of double-stranded nucleic acid molecules may include a second subset of double-stranded nucleic acid molecules comprising a second double-stranded nucleic acid molecule. The second double-stranded nucleic acid molecule may comprise a second sequence corresponding to a second nucleic acid molecule and does not include an added sequence. The first double-stranded nucleic acid molecule may generate a first denaturation profile upon denaturation and the second double-stranded nucleic acid molecule may generate a second denaturation profile upon denaturation. The first and second denaturation profiles may be different and distinguishable. The added sequence may modulate the denaturation profile of the first double-stranded nucleic acid molecule to permit or enhance differentiation between the first and second denaturation profiles.

The system may be configured to implement or may implement any of the methods described elsewhere herein. The system may use any of the devices, reagents, or components described elsewhere herein.

The system may further include a microfluidic device. The microfluidic device may include a plurality of partitions (e.g., an array of partitions). The nucleic acid molecule may be one of a plurality of nucleic acid molecule and the system may provide the plurality of nucleic acid molecules the microfluidic device for partitioning. The system may be further configured to amplify the nucleic acid molecules and subject the nucleic acid molecules to denaturation conditions in the partitions.

The microfluidic devices of the present disclosure may be consumable devices (e.g., designed for a single use, such as analysis or processing of a single sample) or reusable devices (e.g., designed for multiple uses, such as analysis or processing of multiple samples). The choices of materials for inclusion in the device may reflect whether the device may be used one or more times. For example, a consumable device may comprise materials that are less expensive than a reusable device. Similarly, manufacturing processes may be tailored to the use of the device. For example, a fabrication process for a consumable device may involve the production of less waste or involve lower cost manufacturing. A reusable device may be cleanable or sterilizable to facilitate analyses or processing of multiple samples using the same device. For example, a reusable device may comprise materials capable of withstanding high temperatures appropriate for sterilization. A consumable device may or may not comprise such materials.

The microfluidic device may include a fluid flow path. The fluid flow path may include one channel or multiple channels. The fluid flow path may include 1, 2, 3, 4, 5, 6, 8, 10, 12, 15, 20, 25, 30, 40, 50 or more channels. Each channel may be fluidically isolated from one another. Alternatively, or in addition to, the multiple channels may be in fluidic communication with one another. The channel may include a first end and a second end. The first end and second end may be connected to a single inlet port. A channel with a first end and second end connected to a single inlet port may be in a circular or looped configuration such that the fluid entering the channel through the inlet port may be directed through the first end and second end of the channel simultaneously. Alternatively, the first end and second end may be connected to different inlet ports. Alternatively, the first end may be connected to an inlet port and the second end may be connected to an outlet port. Alternatively, the first end may be connected to an inlet port and the second end may be a closed or dead end. The fluid flow path may include a plurality of partitions (e.g., chambers). The fluid flow path or the chambers may not include valves to stop or hinder fluid flow or to isolate the chamber(s).

The device may comprise a long dimension and a short dimension. The long dimension may be less than or equal to about 20 centimeters (cm), 15 cm, 10 cm, 8 cm, 6 cm, 5 cm, 4 cm, 3 cm, 2 cm, 1 cm, or less. The short dimension of the device may be less than or equal to about 10 cm, 8 cm, 6 cm, 5 cm, 4 cm, 3 cm, 2 cm, 1 cm, 0.5 cm, or less. In an example, the dimensions of the device (e.g., microfluidic device) are about 7.5 cm by 2.5 cm. The channel may be substantially parallel to the long dimension of the microfluidic device. Alternatively, or in addition to, the channel may be substantially perpendicular to the long dimension of the microfluidic device (e.g., parallel to the short dimension of the device). Alternatively, or in addition to, the channel may be neither substantially parallel nor substantially perpendicular to the long dimension of the microfluidic device. The angle between the channel and the long dimension of the microfluidic device may be at least about 5°, 10°, 15°, 20°, 30°, 40°, 50°, 60°, 70°, or 90. In an example, the channel is a single long channel. Alternatively, or in addition to, the channel may have bends, curves, or angles. The channel may have a long dimension that is less than or equal to about 100 millimeters (mm), 75 mm, 50 mm, 40 mm, 30 mm, 20 mm, 10 mm, 8 mm, 6 mm, 4 mm, 2 mm, or less. The length of the channel may be bounded by the external length or width of the microfluidic device. The channel may have a depth of less than or equal to about 500 micrometers (μm), 250 μm, 100 μm, 80 μm, 60 μm, 30 μm, 20 μm, 10 μm, or less. The channel may have a cross-sectional dimension (e.g., width or diameter) of less than or equal to about 500 μm, 250 μm, 100 μm, 75 μm, 50 μm, 40 μm, 30 μm, 20 μm, 10 μm, or less.

In some examples, the cross-sectional dimensions of the channel may be about 100 μm wide by about 100 μm deep. In some examples, the cross-sectional dimensions of the channel may be about 100 μm wide by about 80 μm deep. In some examples, the cross-sectional dimensions of the channel may be about 100 μm wide by about 60 μm deep. In some examples, the cross-sectional dimensions of the channel may be about 100 μm wide by about 40 μm deep. In some examples, the cross-sectional dimensions of the channel may be about 100 μm wide by about 20 μm deep. In some examples, the cross-sectional dimensions of the channel may be about 100 μm wide by about 10 μm deep. In some examples, the cross-sectional dimensions of the channel may be about 80 μm wide by about 100 μm deep. In some examples, the cross-sectional dimensions of the channel may be about 60 μm wide by about 100 μm deep. In some examples, the cross-sectional dimensions of the channel may be about 40 μm wide by about 100 μm deep. In some examples, the cross-sectional dimensions of the channel may be about 20 μm wide by about 100 μm deep. In some examples, the cross-sectional dimensions of the channel may be about 10 μm wide by about 100 μm deep. In some examples, the cross-sectional dimensions of the channel may be about 80 μm wide by about 80 μm deep. In some examples, the cross-sectional dimensions of the channel may be about 60 μm wide by about 60 μm deep. In some examples, the cross-sectional dimensions of the channel may be about 40 μm wide by about 40 μm deep. In some examples, the cross-sectional dimensions of the channel may be about 20 μm wide by about 20 μm deep. In some examples, the cross-sectional dimensions of the channel may be about 10 μm wide by about 10 μm deep.

The cross-sectional shape of the channel may be any suitable cross-sectional shape including, but not limited to, circular, oval, triangular, square, or rectangular. The cross-sectional area of the channel may be constant along the length of the channel. Alternatively, or in addition to, the cross-sectional area of the channel may vary along the length of the channel. The cross-sectional area of the channel may vary from about 50% to 150%, 60% to 125%, 70% to 120%, 80% to 115%, 90% to 110%, 95% to 100%, or 98% to 102%. The cross-sectional area of the channel may be less than or equal to about 10,000 micrometers squared (μm²), 7,500 μm², 5,000 μm², 2,500 μm², 1,000 μm², 750 μm², 500 μm², 400 μm², 300 μm², 200 μm², 100 μm², or less.

The channel may have a single inlet or multiple inlets. The inlet(s) may have the same diameter or they may have different diameters. The inlet(s) may have diameters less than or equal to about 2.5 millimeters (mm), 2 mm, 1.5 mm, 1 mm, 0.5 mm, or less.

The device may include a plurality of chambers. The plurality of chambers may be an array of chambers. The device may include a single array of chambers or multiple arrays of chambers, with each array of chambers fluidically isolated from the other arrays. The array of chambers may be arranged in a row, in a grid configuration, in an alternating pattern, or in any other configuration. The microfluidic device be generated on a substrate and the substrate may have at least 1, 2, 3, 4, 5, 10, 15, 20, 30, 40, 50, or more arrays of chambers. In an example, the substrate may include at least 4 arrays of chambers. The substrate may be provided in a consumable plate. The consumable plate may be configured to hold or may hold at least 1, 2, 3, 4, 6, 8, 10, 12, 15, 20, or more substrates. In an example, the consumable plate holds at least 4 substrates. The arrays of chambers may be identical of the arrays of chambers may be different (e.g., have a different number or configuration of chambers). The arrays of chambers may all have the same external dimension (i.e., the length and width of the array of chambers that encompasses all features of the array of chambers) or the arrays of chambers may have different external dimensions. An array of chambers may have a width of less than or equal to about 100 mm, 75 mm, 50 mm, 40 mm, 30 mm, 20 mm, 10 mm, 8 mm, 6 mm, 4 mm, 2 mm, 1 mm, or less. The array of chambers may have a length of greater than or equal to about 50 mm, 40 mm, 30 mm, 20 mm, 10 mm, 8 mm, 6 mm, 4 mm, 2 mm, 1 mm, or less. In an example, the width of an array may be from about 1 mm to 100 mm or from about 10 mm to 50 mm. In an example, the length of an array may be from about 1 mm to 50 mm or from about 5 mm to 20 mm.

The array of chambers may have greater than or equal to about 1,000 chambers, 5,000 chambers, 10,000 chambers, 20,000 chambers, 30,000 chambers, 40,000 chambers, 50,000 chambers, 100,000 chambers, or more. In an example, the microfluidic device may have from about 10,000 to 30,000 chambers. In another example, the microfluidic device may have from about 15,000 to 25,000 chambers. The chambers may be cylindrical in shape, hemispherical in shape, or a combination of cylindrical and hemispherical in shape. Alternatively, or in addition to, the chambers may be cubic in shape. The chambers may have a cross-sectional dimension of less than or equal to about 500 μm, 250 μm, 100 μm, 80 μm, 60 μm, 30 μm, 15 μm, or less. In an example, the chamber has a cross-sectional dimension (e.g., diameter or side length) that is less than or equal to about 250 μm. In another example, the chamber has a cross-sectional dimension (e.g., diameter or side length) that is less than or equal to about 100 μm. In another example, the chamber has a cross-sectional dimension (e.g., diameter or side length) that is less than or equal to about 50 μm.

The depth of the chambers may be less than or equal to about 500 μm, 250 μm, 100 μm, 80 μm, 60 μm, 30 μm, 15 μm, or less. In an example, the chambers may have a cross-sectional dimension of about 30 μm and a depth of about 100 μm. In another example, the chambers may have a cross-sectional dimension of about 35 μm and a depth of about 80 μm. In another example, the chambers may have a cross-sectional dimension of about 40 μm and a depth of about 70 μm. In another example, the chambers may have a cross-sectional dimension of about 50 μm and a depth of about 60 μm. In another example, the chambers may have a cross-sectional dimension of about 60 μm and a depth of about 40 μm. In another example, the chambers may have a cross-sectional dimension of about 80 μm and a depth of about 35 μm. In another example, the chambers may have a cross-sectional dimension of about 100 μm and a depth of about 30 μm. In another example, the chambers and the channel have the same depth. In an alternative embodiment, the chambers and the channel have different depths.

The chambers may have any volume. The chambers may have the same volume or the volume may vary across the microfluidic device. The chambers may have a volume of less than or equal to about 1000 picoliters (pL), 900 pL, 800 pL, 700 pL, 600 pL, 500 pL, 400 pL, 300 pL, 200 pL, 100 pL, 75 pL, 50 pL, 25 pL, or less picoliters. The chambers may have a volume from about 25 pL to 50 pL, 25 pL to 75 pL, 25 pL to 100 pL, 25 pL to 200 pL, 25 pL to 300 pL, 25 pL to 400 pL, 25 pL to 500 pL, 25 pL to 600 pL, 25 pL to 700 pL, 25 pL to 800 pL, 25 pL to 900 pL, or 25 pL to 1000 pL. In an example, the chamber(s) have a volume of less than or equal to 500 pL. In another example, the chambers have a volume of less than or equal to about 250 pL. In another example, the chambers have a volume of less than or equal to about 100 pL.

The volume of channel may be less than, equal to, or greater than the total volume of the chambers. In an example, the volume of the channel is less than the total volume of the chambers. The volume of the channel may be less than or equal to 95%, 90%, 80%, 70%, 60%, 50%, 40%, 30%, 20%, 10%, or less than the total volume of the chambers.

The device may further include a siphon aperture disposed between the channel and the chamber. The siphon aperture may be one of a plurality of siphon apertures connecting the channel to a plurality of chambers. The siphon aperture may be configured to provide fluid communication between the channel and the chamber. The lengths of the siphon apertures may constant or may vary across the device (e.g., microfluidic device). The siphon apertures may have a long dimension that is less than or equal to about 150 μm, 100 μm, 50 μm, 25 μm, 10 μm, 5 μm, or less. The depth of the siphon aperture may be less than or equal to about 50 μm, 25 μm, 10 μm, 5 μm, or less. The siphon apertures may have a cross-sectional dimension of less than or equal to about 50 μm, 40 μm, 30 μm, 20 μm, 10 μm, 5 μm, or less.

The cross-sectional shape of the siphon aperture may be any suitable cross-sectional shape including, but not limited to, circular, oval, triangular, square, or rectangular. The cross-sectional area of the siphon aperture may be constant along the length of the siphon aperture. Alternatively, or in addition to, the cross-sectional area of the siphon aperture may vary along the length of the siphon aperture. The cross-sectional area of the siphon aperture may be greater at the connection to the channel than the cross-sectional area of the siphon aperture at the connection to the chamber. Alternatively, the cross-sectional area of the siphon aperture at the connection to the chamber may be greater than the cross-sectional area of the siphon aperture at the connection to the channel. The cross-sectional area of the siphon aperture may vary from about 50% to 150%, 60% to 125%, 70% to 120%, 80% to 115%, 90% to 110%, 95% to 100%, or 98% to 102%. The cross-sectional area of the siphon aperture may be less than or equal to about 2,500 μm², 1,000 μm², 750 μm², 500 μm², 250 μm², 100 μm², 75 μm², 50 μm², 25 μm², or less. The cross-sectional area of the siphon aperture at the connection to the channel may be less than or equal to the cross-sectional area of the channel. The cross-sectional area of the siphon aperture at the connection to the channel may be less than or equal to about 98%, 95%, 90%, 85%, 80%, 75%, 70%, 60%, 50%, 40%, 30%, 20%, 10%, 5%, 1%, 0.5%, or less of the cross-sectional area of the channel. The siphon apertures may be substantially perpendicular to the channel. Alternatively, or in addition to, the siphon apertures are not substantially perpendicular to the channel. An angle between the siphon apertures and the channel may be at least about 5°, 10°, 15°, 20°, 30°, 40°, 50°, 60°, 70°, or 90°.

The microfluidic device may be configured to permit pressurized off-gassing or degassing of the channel, chamber, siphon aperture, or any combination thereof. Pressurized off-gassing or degassing may be provided by a film or membrane configured to permit pressurized off-gassing or degassing. Alternatively, or in addition to, pressurized off-gassing or degassing may be provided by a second channel (e.g., off-gas channel) disposed adjacent to the chamber, channels, or both. The second channel may permit pressurized off-gassing or degassing above a pressure threshold. The film or membrane may be permeable to gas above a pressure threshold. The film or membrane may not be permeable to (e.g., is impermeable or substantially impermeable to) liquids such as, but not limited to, aqueous fluids, oils, or other solvents. The channel, the chamber, the siphon aperture, or any combination thereof may comprise the film or membrane. In an example, the chamber comprises the gas permeable film or membrane and the channel or siphon aperture does not comprise the gas permeable film or membrane. In another example, the chamber and siphon aperture comprises the gas permeable film or membrane and the channel does not comprise the gas permeable film or membrane. In another example, the chamber, channel, and siphon aperture comprise the gas permeable film or membrane.

The film or membrane may be a thin film. The film or membrane may be a polymer. The film may be a thermoplastic film or membrane. The film or membrane may not comprise an elastomeric material. The gas permeable film or membrane may cover the fluid flow path, the channel, the chamber, or any combination thereof. In an example, the gas permeable film or membrane covers the chamber. In another example, the gas permeable film or membrane covers the chamber and the channel. The gas permeability of the film may be induced by elevated pressures. The thickness of the film or membrane may be less than or equal to about 500 micrometers (μm), 250 μm, 200 μm, 150 μm, 100 μm, 75 μm, 50 μm, 25 μm, or less. In an example, the film or membrane has a thickness of less than or equal to about 100 μm. In another example, the film or membrane has a thickness of less than or equal to about 50 μm. In another example, the film or membrane has a thickness of less than or equal to about 25 μm. The thickness of the film or membrane may be from about 0.1 μm to about 200 μm, 0.5 μm to 150 μm, or 25 μm to 100 μm. In an example, the thickness of the film or membrane is from about 25 μm to 100 μm. The thickness of the film may be selected by manufacturability of the film, the air permeability of the film, the volume of each chamber or partition to be out-gassed, the available pressure, or the time to complete the partitioning or digitizing process.

The film or second channel may be configured to employee different permeability characteristics under different applied pressure differentials. For example, the thin film or second channel may be gas impermeable at a first pressure differential (e.g., low pressure) and at least partially gas permeable at a second pressure differential (e.g., high pressure). The first pressure differential (e.g., low pressure differential) may be less than or equal to about 8 pounds per square inch (psi), 6 psi, 4 psi, 2 psi, 1 psi, or less. In an example, the film or membrane is substantially impermeable to gas at a pressure differential of less than 4 psi. The second pressure differential (e.g., high pressure differential) may be greater than or equal to about 1 psi, 2 psi, 4 psi, 6 psi, 8 psi, 10 psi, 12 psi 14 psi, 16 psi, 20 psi, or more. In an example, the film or membrane is substantially gas permeable at a pressure of greater than or equal to 4 psi.

The system may include a holder configured to receive or hold the microfluidic device. The holder may be a shelf, receptacle, or stage for holding the device. In an example, the holder is a transfer stage. The transfer stage may be configured input the microfluidic device, hold the microfluidic device, and output the microfluidic device. The microfluidic device may be any device described elsewhere herein. The transfer stage may be stationary in one or more coordinates. Alternatively, or in addition to, the transfer stage may be capable of moving in the X-direction, Y-direction, Z-direction, or any combination thereof. The transfer stage may be capable of holding a single microfluidic device. Alternatively, or in addition to, the transfer stage may be capable of holding at least 2, 3, 4, 5, 6, 7, 8, 9, 10, or more microfluidic devices.

The system may include a processing unit. The processing unit may include a pneumatic module, vacuum module, or any combination thereof. The processing unit may be configured to amplify the nucleic acid molecules in the plurality of partitions or chambers. The processing unit may be configured to be in fluid communication with the inlet port(s) of the microfluidic device. The processing unit may have multiple connection points capable of connecting to multiple inlet port(s). The processing unit may be able to fill, backfill, and partition a single array of chambers at a time or multiple arrays of chambers in tandem. The processing unit may be a pneumatic module combined with a vacuum module. The processing unit may provide increased pressure to the microfluidic device or provide vacuum to the microfluidic device for pressurized off-gassing or degassing.

The system may further comprise a thermal unit. The thermal unit may be operatively coupled to one or more processors. The thermal unit may be configured to provide resistive heating, radiative heating, conductive heating, convective heating, thermoelectric heating, or any combination thereof. In an example, the thermal unit comprises a thermoelectric temperature control unit. The thermal unit may be configured to be in thermal communication with the chambers of the microfluidic devices. The thermal unit may be configured to control the temperature of a single array of chambers or to control the temperature of multiple arrays of chambers to permit thermal denaturation of the nucleic acid molecules. An array of chambers may be individually addressable by the thermal unit. For example, thermal unit may perform the same thermal program across all arrays of chambers or may perform different thermal programs with different arrays of chambers. The thermal unit may be in thermal communication with the microfluidic device or the chambers of the microfluidic device. The thermal unit may heat or cool the microfluidic device. One or more surfaces of the microfluidic device may be in direct contact with the thermal unit. Alternately, or in addition to, a thermally conductive material may be disposed between the thermal module and the microfluidic device. The thermal unit may maintain the temperature across a surface of the microfluidic device such that the variation is less than or equal to about 2° C., 1.5° C., 1° C., 0.9° C., 0.8° C., 0.7° C., 0.6° C., 0.5° C., 0.4° C., 0.3° C., 0.2° C., 0.1° C., or less. The thermal unit may maintain a temperature of a surface of the microfluidic device that is within about plus or minus 0.5° C., 0.4° C., 0.3° C., 0.2° C., 0.1° C., 0.05° C., or closer to a temperature set point.

The system may further include a detection unit. The detection module may provide electronic or optical detection. In an example, the detection unit is an optical unit providing optical detection. The optical unit may be configured to emit and detect multiple wavelengths of light. Emission wavelengths may correspond to the excitation wavelengths of the indicator and amplification probes used. The emitted light may include wavelengths with a maximum intensity around about 450 nm, 500 nm, 525 nm, 550 nm, 575 nm, 600 nm, 625 nm, 650 nm, 675 nm, 700 nm, or any combination thereof. Detected light may include wavelengths with a maximum intensity around about 500 nm, 525 nm, 550 nm, 575 nm, 600 nm, 625 nm, 650 nm, 675 nm, 700 nm, or any combination thereof. The optical unit may be configured to emit more than one, two, three, four, or more wavelengths of light. The optical unit may be configured to detect more than one, two, three, four, or more wavelengths of light. One emitted wavelength of light may correspond to the excitation wavelength of an indicator molecule. Another emitted wavelength of light may correspond to the excitation wavelength of an amplification probe. One detected wavelength of light may correspond to the emission wavelength of an indicator molecule. Another detected wavelength of light may correspond to an amplification probe used to detect a reaction within the chambers. The optical unit may be configured to image sections of an array of chambers. Alternatively, or in addition to, the optical unit may image an entire array of chambers in a single image. In an example, the optical unit is configured to take video of the device.

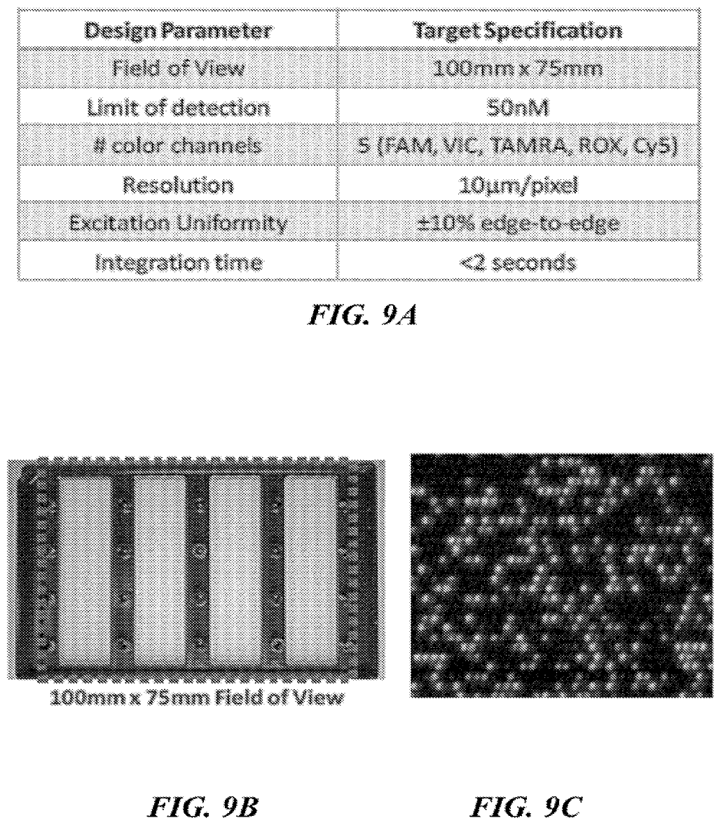

The detection unit may include a stationary imaging unit configured to capture data from the consumable plate. FIG. 9A shows example design parameters for the imaging unit. The imaging unit may be configured to image at least a portion of the plurality of chambers (e.g., consumable device) or the entire consumable device. The amount of the consumable device imaged by the imaging unit may be determined by the filed of view of the imaging device. The imaging unit may have a filed of view of greater than or equal to 15 millimeters (mm) by 15 mm, 15 mm by 20 mm, 15 mm by 25 mm, 15 mm by 50 mm, 15 mm by 75 mm, 15 mm by 100 mm, 20 mm by 20 mm, 20 mm by 25 mm, 20 mm by 50 mm, 20 mm by 75 mm, 20 mm by 100 mm, 25 mm by 25 mm, 25 mm by 50 mm, 25 mm by 75 mm, 25 mm by 100 mm, 50 mm by 50 mm, 50 mm by 75 mm, 50 mm by 100 mm, 75 mm by 75 mm, 75 mm by 100 mm, or 100 mm by 100 mm. In an example, the field of view of the imaging unit is greater than or equal to about 15 mm by 15 mm. In another example, the field of view of the imaging unit is greater than or equal to about 50 mm by 75 mm. In another example, the field of view of the imaging unit is greater than or equal to about 75 mm by 100 mm. The imaging module may include a stationary camera with a large field of view that can capture data from the entire consumable plate with a single image. FIG. 9B shows an example field of view for imaging an entire example consumable plate and FIG. 9C shows an example image taken from an example imaging unit. To account for mechanical tolerance and minimize spherical aberration, the field of view may be 100 mm by 75 mm, which may provide imaging for sixteen partition units of the consumable with sufficient margin. Other engineering efforts may include minimizing moving parts for better stability and repeatability and optimizing for cost.

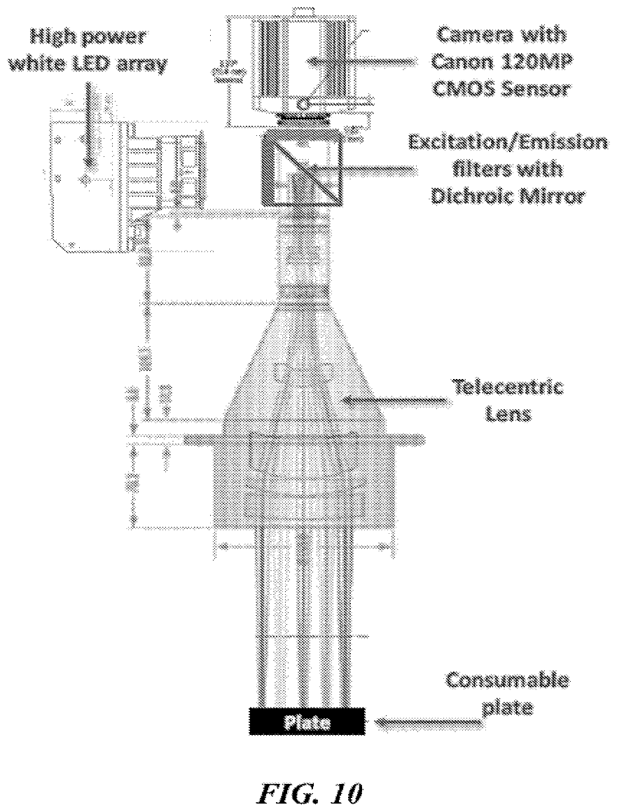

The imaging unit may include a lens and a sensor. With a 100 mm×75 mm field of view, many lenses may not provide image quality and uniformity sufficient for dPCR. The lens may be a telecentric lens. Use of telecentric lens may eliminate or reduce parallax error. For example, zero angular field of view may minimize image distortion and improve uniformity, as well as exhibit sharp focus transition to permit partition finding. On the sensor side, to achieve 10 μm/pixel resolution over the 100 mm×75 mm field of view, large area complementary metal-oxide-semiconductor (CMOS) sensor with greater than one inch diagonal and over 100 million pixels may be used. For example, the Canon 120MP CMOS sensor may be a viable option to achieve the resolution and limit of detection. For uniform excitation, two approaches may be used, oblique illumination from the side of the plate, or upright epi-illumination through a telecentric lens. For example, oblique illumination may be provided by a high power density light emitting diodes (LEDs). Two different LED excitation strategies may be used: High brightness white light LED and color-specific LEDs. To support the five most common PCR dyes FAM, VIC, TAMRA, ROX and Cy5, excitation and emission filters as well as dichroic mirror (unless oblique illumination architecture was selected) may be used. FIG. 10 shows an example imaging unit. The imaging unit may include a high power LED array, camera with CMOS sensor, one or more excitation and emission filters, or telecentric lens. The excitation and emission filters may be disposed between the camera and the telecentric lens. The telecentric lens may be disposed between the camera and the consumable plate. To confirm that the optical module meets the target specification, dyes of various concentrations (e.g., 150 nanomolar (nM), 100 nM, 50 nM and 0 nM as the negative control) may be loaded into the plate. The plate may be images with the breadboard, and the signal to noise ration may be computed based on ASTM E579 standard (fluorescence limit of detection).

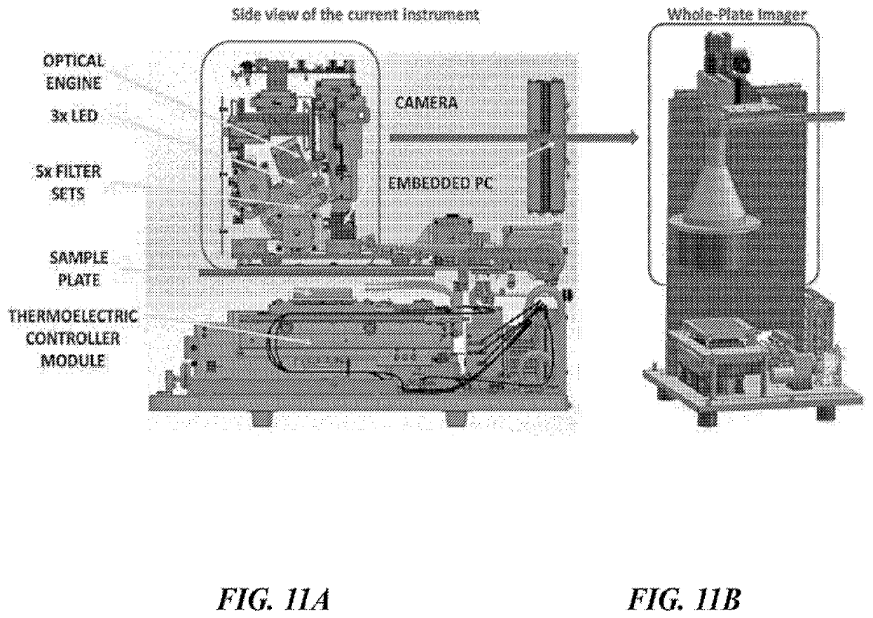

FIGS. 11A and 11B show an example schematics of an integrated system for dPCR. FIG. 11A shows an example side view of an integrated system for dPCR. The integrated system may include a thermal control module for PCR, pneumatic control module for reagent digitization and optical module for four-color scanning of the whole plate. The components of the system may be vertically integrated. Four-color scanning may be provided by an optical engine with four channels, each channel configured to collect a different wavelength of light. The instrument may be modular (e.g., all units may be physically independent from each other), which permits units to be replaced or exchanges without impacting other subsystems. A modular design may reduce the time and risks for instrument development. FIG. 11B shows an example of a whole plate imaging unit. The use of an imaging unit with a long working distance, the scanning motion may be eliminated and the overall width of the system may be reduced compared to a similar instrument with a scanning imaging unit.

The system may further include a robotic arm. The robotic arm may move, alter, or arrange a position of the microfluidic device. Alternatively, or in addition to, the robotic arm may arrange or move other components of the system (e.g., processing unit or detection unit). The detection unit may include a camera (e.g., a complementary metal oxide semiconductor (CMOS) camera) and filter cubes. The filter cubes may alter or modify the wavelength of excitation light or the wavelength of light detected by the camera. The processing unit may comprise a manifold (e.g., pneumatic manifold) or one or more pumps. The manifold may be in an upright position such that the manifold does not contact the microfluidic device. The upright position may be used when loading or imaging the microfluidic device. The manifold may be in a downward position such that the manifold contacts the microfluidic device. The manifold may be used to load fluids (e.g., samples and reagents) into the microfluidic device. The manifold may apply a pressure to the microfluidic device to hold the device in place or to prevent warping, bending, or other stresses during use. In an example, the manifold applies a downward pressure and holds the microfluidic device against the thermal unit.

The system may further include one or more computer processors. The one or more computer processors may be operatively coupled to the processing unit, holder, thermal unit, detection unit, robotic arm, or any combination thereof. In an example, the one or more computer processors is operatively coupled to the processing unit. The one or more computer processors may be individually or collectively programmed to direct the processing unit to partition the nucleic acid molecules, amplify the nucleic acid molecules, denature the nucleic acid molecule, or any combination thereof. The one or more computer processors may be individually or collectively programmed or otherwise configured to direct the detection unit to collect signals indicative of denaturation of the nucleic acid molecules. The one or more computer processors may be individually or collectively programmed or otherwise configured to generate a denaturation profile of the nucleic acid molecule, use the denaturation profile to identify or quantify the analyte, or any combination thereof.

The one or more processors may be configured to implement one or more software programs. The software may be a web-based user interface. A web-based user interface may allow for implementation of continuous improvements as well as facilitate remote service and support. Three user interface modules may be used, a protocol engine, image processing, and digital melt curve analysis. The protocol engine may provide for variability in front end processes (e.g., dPCR) and backend processes (e.g., dMCA). The protocol engine may permit the user to change reagent digitization protocols (e.g., pressure and time), thermal cycling protocol (e.g., number of cycles, temperature and time), number of images to be acquired, melt process protocol (e.g., temperature start and end point, ramp rate and interval), and imaging protocol (e.g., LED power, filter set, and exposure time). The protocols may be compiled and loaded onto the instrument so that the instrument runs independent of an active and connected external computer. For image processing, raw images may be optimized for analysis and displayed. The user may be able to use various tools such as image stacking (e.g., multiple colors overlays), image arithmetic (e.g., subtraction), array identification assignment, and automated array stenciling. The output may be an interpretable .txt or .csv file with parameters for each partition: Index, unit, row, column, total intensity, and Quality Control (QC) flags. The imaging software may be used for automated segmentation of 320,000 chambers (16 units×20 k chambers per unit), cropping out unused sensor areas, and normalization of chamber-to-chamber signal variations caused by potential non-uniform heating or illumination across the field of view. Digital melt curve analysis may provide partition counting, Poisson statistical models, dilution factor considerations, real-time curves, melt curves or derivative of melt curves, and experimental notes for export in the form of reports. Quality control tools such as partition review (e.g., examination of raw images) and feature flagging may be implemented so that the user can manually exclude known false features (e.g., identified by outlier values of local background or reporter channel intensity). Machine learning algorithm t-SNE (e.g., t-distributed stochastic neighbor embedding) may be used to cluster similar melt curves. As each plate may offer over 300,000 melt curves, by running four plates with identical experiment, over one million training data set may be provided to the instrument to greatly improve the calling accuracy. In addition, the web-based application may permit data analysis and algorithm development to be ongoing, continuously reviewed, and updated with the latest features and application tools.

### Computer Systems

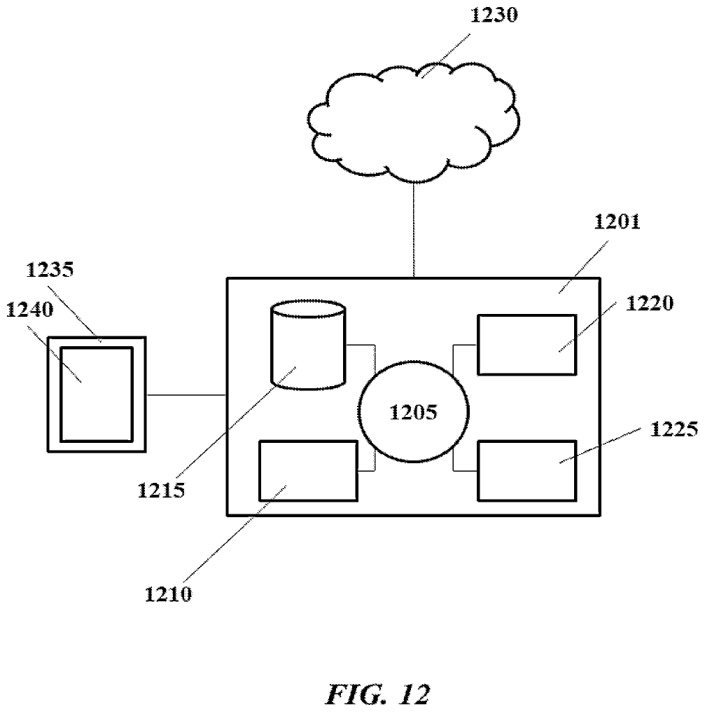

The present disclosure provides computer systems that are programmed to implement methods of the disclosure. FIG. 12 shows a computer system 1201 that is programmed or otherwise configured to implement the methods described elsewhere herein for analysis and identification of an analyte. The computer system 1201 can regulate various aspects of processing and analysis of analytes (e.g., nucleic acid molecules), such as, for example, partitioning a sample (e.g., into chambers), amplifying the sample, denaturing the sample, and detecting signals during denaturation. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user (e.g., cell phone, laptop computer, desktop computer, or other user device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, system operating parameters, status of one or more of the system, subsystems, or various units (e.g., detection unit, flow unit, etc.), or analysis parameters and results. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, process denaturation signals to provide identification or quantification of an analyte (e.g., nucleic acid molecule).

### EXAMPLES

### Example 1: Multiplexing Assay

The microfluidic array consumable and integrated system is used for a proof-of-concept multiplexing assay for Spinal Muscular Atrophy (SMA) newborn screening integrating SMN1 copy number (e.g., gene found in greater than 90% of SMA cases), SMN2 copy number (e.g., gene significant in prognosis), and RPPH1 (reference gene) into a single assay. The dMCA assay uses three amplicons targeting SMN1, SMN2, and RPPH2, ranging from 50-75 base pairs and generated by five primers. Due to the high homologous sequences between SMN1 and SMN2, allele specific primers targeting the splicing site in Exon7 are used to distinguish the two genes. SMN1 includes adenine at nucleotide 804 while SMN2 includes thymine at the same position. The SMN1 “A” allele-specific primer does not amplify PCR products from SMN2 template, and vice versa for the SMN2 “T” allele-specific primer. Additional variable length of tails at the 5′ end of the forward primers may increase the melt temperature separation to further differentiate the two targets. Two primers are used to amplify the highly conserved RPPH1 gene which has two copies as internal copy number control. The design was verified with the μMelt software developed at University of Utah where three distinct melt peaks is predicted (71° C., 76° C. and 81° C.). In addition, primer specificities using NCBI Primer-Blast and UCSC In-Silico PCR tools were also conducted.

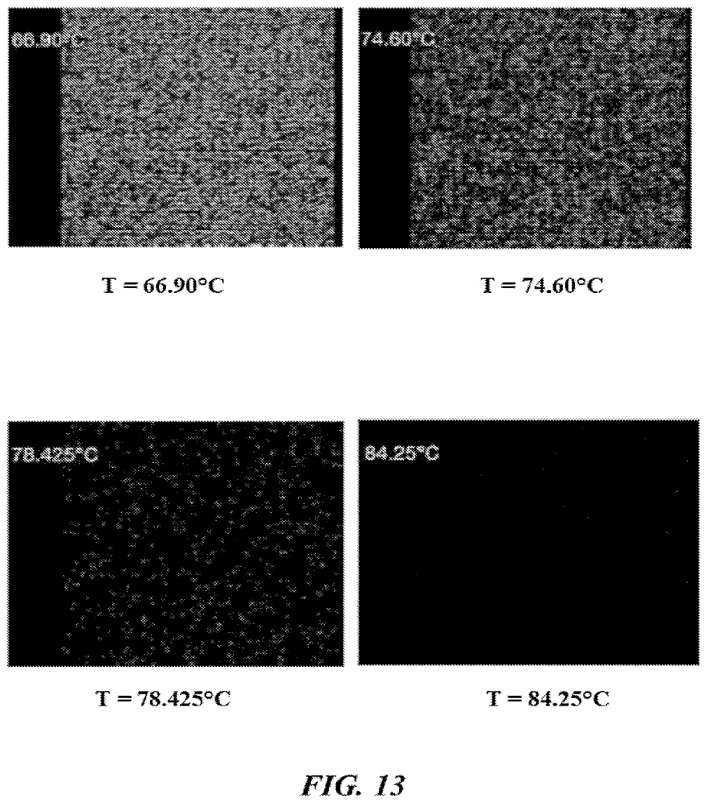

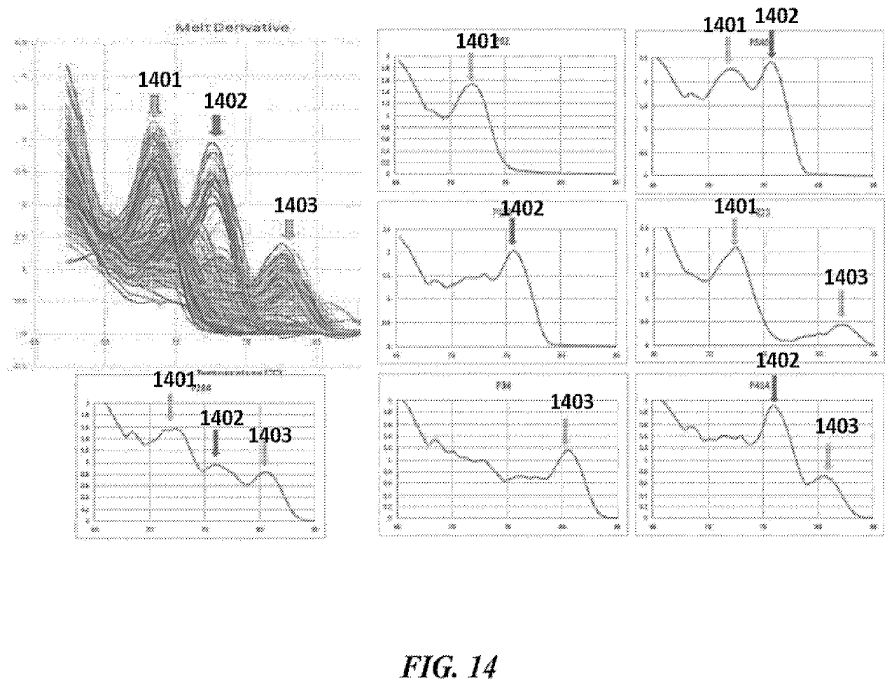

The primer sets include EvaGreen double-strand DNA intercalating dyes and 2×MasterMix from Biotium. Human genomic DNA (gDNA) from MilliporeSigma is the target. The dMCA reagent mix is prepared with a final gDNA concentration of approximately one copy per partition. After 40 cycles of thermal cycling between 59° C. and 95° C., amplicon melting is performed from 59° C. to 95° C. at a ramp rate of 0.2° C. per second resolution. FIG. 13 shows example fluorescent images acquired at four different temperatures, demonstrating the melting of three amplicons from 65° C. to 85° C. The example melt curves (derivatives of the fluorescence signals) from 200 randomly selected partitions are shown in FIG. 14. The example melt curves show multiple melt temperatures representing the three different targets, SMN1 1401, SMN2 1402, and RPPH1 1403.

### Example 2: Broad-Scale Species Identification

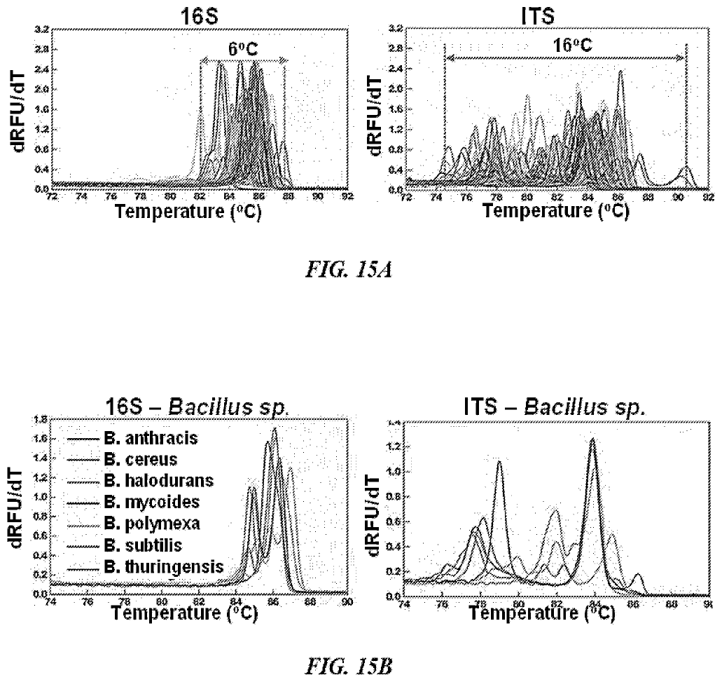

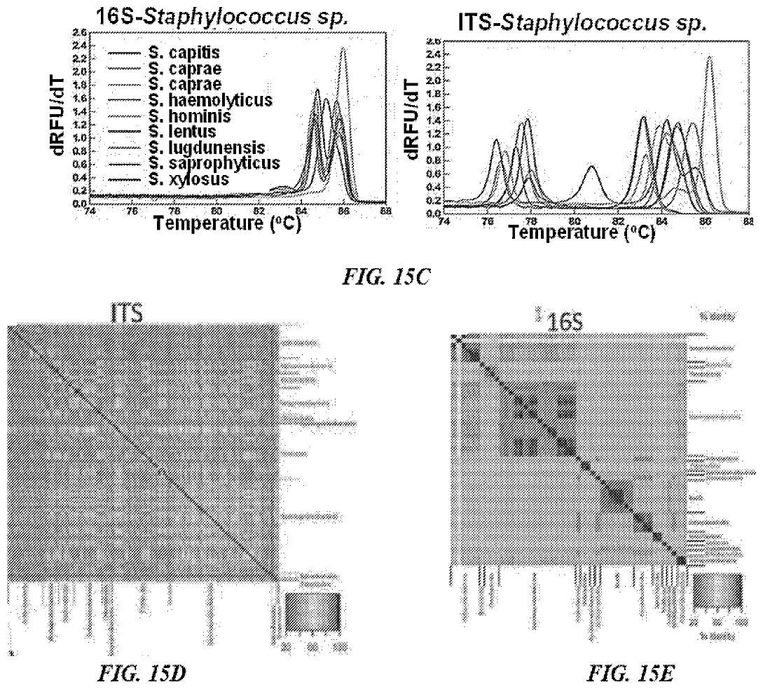

Broad PCR using a single pair of conserved primers may enables unbiased amplification of potential sequence variants of interest. Coupling it with melt curve analysis (MCA) may allow concurrent “fingerprinting” of the amplicon sequences based on their melt profile. Although melt curves derived from sixteen base long amplicons (16S) confer more biphasic curve profiles than those from shorter amplicons, the narrow melting temperature range and limited profile diversity may constrain use for broad-scale species-level identification. In MCA, melting temperature and curve shapes are functions of sequence, percent GC content, length, melt domains, and sequence complementarity. The bacterial internal transcribed spacer (ITS) sequence between the 16S-23S rDNA may be less evolutionarily constrained than its flanking genes and can enable enhanced species-level phylogenetic discrimination. Moreover, its unique intragenomic sequence heterogeneity with multiple melt domains contributes to complex melt curve shapes, making it well-suited as a single phylogenetic locus to simplify assay format. Using an archived library of 89 different bacterial species to perform qPCR-HRM, it is demonstrated that ITS amplicons generate rich melt curve profiles, with multiple peaks and a much wider melting temperature range, as compared to 16S amplicons, as shown in FIG. 15A, for enhanced species identification. Close members of the same genus may be indistinguishable by their 16S curves and may be are visually distinct based on ITS curves, see FIGS. 15B and 15C showing examples for Bacillus species and Staphylococcus species, respectively. Preliminary sequence analysis of the ITS rDNA suggests sufficient discriminatory power for target species, as shown in the heat map of FIG. 15D. The same analysis applied to 16S, as shown in FIG. 15E, may achieve significantly lower discriminatory power. Methods have also been developed (e.g., Naïve Bayes (nB) curve classification algorithm) for statistical interpretation of melting curve data and can achieve over 95% accuracy in differentiating 89 bacterial species in the library using leave-one-out cross-validation. Using the broad-based PCR of the ITS region followed by melt curve analysis, a developed database containing over 2,000 “molecular fingerprints” obtained from patients at Stanford Hospital, may be used to benchmark the performance of the dMCA platform.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for nucleic acid identification, comprising:
(a) using a plurality of nucleic acid molecules to generate, in a plurality of chambers, a plurality of double-stranded nucleic acid molecules, wherein: (i) a first subset of said plurality of double-stranded nucleic acid molecules comprises a first double-stranded nucleic acid molecule comprising a first sequence corresponding to a first nucleic acid molecule of said plurality of nucleic acid molecules and an added sequence; and (ii) a second subset of said plurality of double-stranded nucleic acid molecules comprises a second double-stranded nucleic acid molecule comprising a second sequence corresponding to a second nucleic acid molecule of said plurality of nucleic acid molecules and does not comprise said added sequence;
(b) denaturing double-stranded nucleic acid molecules of said plurality of double-stranded nucleic acid molecules;
(c) detecting signals indicative of said denaturing to generate a plurality of denaturation profiles, wherein:
i. a first denaturation profile of said plurality of denaturation profiles is derived from denaturation of said first double-stranded nucleic acid molecule;
ii. a second denaturation profile of said plurality of denaturation profiles is derived from denaturation of said second double-stranded nucleic acid molecule; and
iii. said first denaturation profile and said second denaturation profile are different; and
(d) processing said plurality of denaturation profiles to identify a nucleic acid molecule of said plurality of nucleic acid molecules.

2. The method of claim 1, further comprising, prior to (a), providing said plurality of nucleic acid molecules and a plurality of forward primers to said plurality of chambers.

3. The method of claim 2, wherein said plurality of forward primers comprises (i) a first forward primer comprising a first region complementary to at least a portion of said first nucleic acid molecule and a second region that is not complementary to said first nucleic acid molecule and corresponds to said added sequence and (ii) a second forward primer complementary to at least a portion of said second nucleic acid molecule.

4. The method of claim 3, wherein said plurality of forward primers are not universal primers.

5. The method of claim 3, further comprising, prior to (a), subjecting said plurality of forward primers to primer extension reactions to generate a plurality of first extension products.

6. The method of claim 5, further comprising, prior to (a), contacting said plurality of first extension products with a plurality of reverse primers.

7. The method of claim 6, wherein said plurality of reverse primers are universal primers.

8. The method of claim 6, further comprising, prior to (a), subjecting said plurality of reverse primers to primer extension reactions to generate a plurality of second extension products.

9. The method of claim 8, wherein said plurality of second extension products are said plurality of double-stranded nucleic acid molecules.

10. The method of claim 1, further comprising imaging at least a portion of said plurality of chambers to detect said signals.

11. The method of claim 10, further comprising imaging said plurality of chambers to detect said signals.

12. The method of claim 1, further comprising subjecting said plurality of double-stranded nucleic acid molecules to controlled heating to denature said double-stranded nucleic acid molecules.

13. The method of claim 1, wherein said double-stranded nucleic acid molecules comprise intercalating dyes from which said signals are derived.

14. The method of claim 13, wherein said double-stranded nucleic acid molecules comprise a plurality of different intercalating dyes from which said signals are derived.

15. The method of claim 1, wherein said signals are optical signals.

16. The method of claim 1, wherein a chamber of said plurality of chambers has a volume of less than or equal to about 500 picoliters.

17. The method of claim 16, wherein said volume of said chamber is less than or equal to about 250 picoliters.

18. The method of claim 1, wherein said plurality of chambers comprises greater than or equal to about 1,000 chambers.

19. The method of claim 18, wherein said plurality of chambers comprises greater than or equal to about 10,000 chambers.

20. A system for nucleic acid identification, comprising:
a detection unit configured to collect and process signals for identification of nucleic acid molecules; and
one or more processors operatively coupled to said detection unit, wherein said one or more processors are individually or collectively programmed or otherwise configured to:
(i) use a plurality of nucleic acid molecules to generate, in a plurality of chambers, a plurality of double-stranded nucleic acid molecules, wherein: (i) a first subset of said plurality of double-stranded nucleic acid molecules comprises a first double-stranded nucleic acid molecule comprising a first sequence corresponding to a first nucleic acid molecule of said plurality of nucleic acid molecules and an added sequence; and (ii) a second subset of said plurality of double-stranded nucleic acid molecules comprises a second double-stranded nucleic acid molecule comprising a second sequence corresponding to a second nucleic acid molecule of said plurality of nucleic acid molecules and does not comprise said added sequence;
(ii) denature double-stranded nucleic acid molecules of said plurality of double-stranded nucleic acid molecules;
(iii) detect signals indicative of said denaturing to generate a plurality of denaturation profiles, wherein:
(A) a first denaturation profile of said plurality of denaturation profiles is derived from denaturation of said first double-stranded nucleic acid molecule;
(B) a second denaturation profile of said plurality of denaturation profiles is derived from denaturation of said second double-stranded nucleic acid molecule; and
(C) said first denaturation profile and said second denaturation profile are different; and
(iv) processing said plurality of denaturation profiles to identify a nucleic acid molecule of said plurality of nucleic acid molecules.

21. The system of claim 20, wherein a chamber of said plurality of chambers has a volume of less than or equal to about 500 picoliters.

22. The system of claim 21, wherein said volume of said chamber is less than or equal to about 250 picoliters.

23. The system of claim 20, wherein said plurality of chambers comprises greater than or equal to about 1,000 chambers.

24. The system of claim 23, wherein said plurality of chambers comprises greater than or equal to about 10,000 chambers.

25. The system of claim 20, wherein said detection unit is configured to image at least a portion of said plurality of chambers.

26. The system of claim 25, wherein said detection unit is configured to image said plurality of chambers.

27. The system of claim 25, wherein said detection unit comprises a camera with a field of view of greater than or equal to about 15 millimeters (mm) by about 15 mm.

28. The system of claim 27, wherein said field of view that is greater than or equal to about 50 mm by about 75 mm.

29. The system of claim 20, wherein said detection unit comprises a camera comprising a complementary metal-oxide-semiconductor (CMOS) sensor.

30. The system of claim 29, wherein said detection unit further comprises a telecentric lens disposed between said camera and said plurality of chambers.

31. The system of claim 20, wherein said detection unit comprises an optical unit configured to collect optical signals.

32. The system of claim 31, wherein said optical unit comprises greater than or equal to four channels, each channel configured to collect a different wavelength of light.

33. The system of claim 20, wherein said system is configured to receive a substrate comprising a plurality of chamber arrays, and wherein a chamber array of said plurality of chamber arrays comprises said plurality of chambers.

34. The system of claim 33, wherein said substrate comprises at least four chamber arrays.

35. The system of claim 33, wherein said chamber array is fluidically isolated from another chamber array.

36. The system of claim 33, wherein said system is configured to receive a plate, and wherein said plate is configured to retain a plurality of substrates comprising said substrate.

37. The system of claim 20, further comprising a thermal unit operatively coupled to said one or more processors, wherein said thermal unit is configured to control a temperature of said plurality of chambers.

38. The system of claim 37, wherein said one or more processors directs said thermal unit to subject said plurality of chambers to controlled heating to denature said double-stranded nucleic acid molecules.

39. The system of claim 37, wherein said thermal unit comprises a thermoelectric temperature control unit.
